(19) United States
(12) Patent Application Publication
(10) Pub. No.: US 2026/0224435 A1
(43) Pub. Date: Aug. 6, 2026

(54) ADAPTIVE GASTRIC VIBRATIONAL THERAPY SYSTEM WITH CHEWING DETECTION AND PHYSIOLOGICAL MONITORING

(71) Applicant: Appetec, Inc., Macon, GA (US)

(72) Inventor: Shahriar Sedghi, Macon, GA (US)

(73) Assignee: Appetec, Inc., Macon, GA (US)

(21) Appl. No.: 19/543,775

(22) Filed: Feb. 18, 2026

Related U.S. Application Data
(63) Continuation-in-part of application No. 19/043,337, filed on Jan. 31, 2025.

(51) Int. Cl. A61H 23/02; A61B 5/00; A61B 5/01; A61B 5/024; A61B 5/107; A61B 5/11; A61B 5/145; A61F 5/00

(52) U.S. Cl. CPC A61H 23/02; A61B 5/01; A61B 5/02405; A61B 5/107; A61B 5/1126; A61B 5/14503; A61B 5/14532; A61B 5/14546; A61B 5/4035; A61B 5/4836; A61B 5/4869; A61B 5/7235; A61B 5/7282; A61F 5/0003; A61B 2562/0204; A61B 2562/0219; A61H 2201/5012; A61H 2205/083; A61H 2230/045; A61H 2230/085; A61H 2230/203; A61H 2230/505; A61H 2230/855

(57) ABSTRACT

A satiety enhancement system for adaptive control of gastric satiety therapy includes a gastric vibrational therapy device configured to deliver non-invasive mechanical stimulation to a gastric region of a user, a chewing detection sensor configured to monitor eating behavior of the user and generate chewing data indicative of a chewing cadence, a physiological monitoring device configured to acquire physiological data from the user, and a controller in communication with the gastric vibrational therapy device, the chewing detection sensor, and the physiological monitoring device. The controller is configured to receive the chewing data from the chewing detection sensor, receive the physiological data from the physiological monitoring device, determine a target chewing cadence based on the physiological data, calculate a deviation between the chewing cadence and the target chewing cadence, and modulate operational parameters of the gastric vibrational therapy device based on the calculated deviation.

### CROSS-REFERENCE TO RELATED APPLICATIONS

This application is a continuation-in-part of U.S. Application No. 19/043,337, titled AI-Integrated External Gastric Stimulation System for Appetite Control and Behavioral Modification, filed Jan. 31, 2025, which is hereby incorporated by reference in its entirety.

### FIELD OF INVENTION

The present disclosure relates to systems and methods for enhancing satiety and improving metabolic control, and more particularly to a satiety enhancement system integrating a gastric vibrational therapy device, a chewing detection sensor, and a physiological monitoring device with a controller configured to determine a target chewing cadence, calculate deviations from actual chewing cadence, and modulate operational parameters of the gastric vibrational therapy device for personalized closed-loop therapeutic intervention.

### BACKGROUND OF THE INVENTION

Traditional approaches to appetite control and weight management rely on manual food logging applications and retrospective calorie counting that provide limited real-time intervention capabilities during eating episodes. These conventional systems typically operate through user-initiated data entry and fixed dietary guidelines that lack the capability to monitor physiological state, detect eating behavior patterns, or adapt therapeutic interventions through closed-loop control processes. The absence of integrated chewing detection and gastric stimulation frameworks in existing systems constrains their ability to provide both behavioral monitoring and active therapeutic intervention simultaneously.

Current eating pace feedback devices often struggle to create effective satiety enhancement solutions while maintaining user compliance and physiological responsiveness. Many existing systems focus primarily on visual or auditory cues to slow eating, without incorporating sensing modalities that can process real-time chewing data including cadence analysis, bite frequency monitoring, and swallow event detection alongside physiological inputs comprising glucose levels and autonomic nervous system state. This approach results in passive feedback systems that fail to provide therapeutic benefit when users ignore cues or cannot maintain compliance with recommended eating behaviors.

Continuous glucose monitoring systems and activity trackers frequently operate through independent data collection where glucose tracking, sleep monitoring, activity measurement, and dietary assessment function separately without integration layers. This fragmentation leads to inefficiencies in metabolic management, missed opportunities for coordinated intervention based on multiple physiological parameters, and inability to provide unified therapeutic response through adaptive device control. The lack of food property analysis adapted to specific meal contexts for determining appropriate eating pace targets further limits flexibility and effectiveness in satiety enhancement systems.

Existing therapeutic approaches to appetite regulation typically focus on narrow use cases such as basic calorie restriction or simple meal timing that lack contextual understanding and physiological adaptability. These implementations cannot develop meaningful intervention protocols through multi-modal data integration, maintain consistent therapeutic efficacy across varying metabolic states including different glucose levels and autonomic conditions, or coordinate between chewing detection sensors capturing eating behavior and controllers generating adaptive stimulation parameters. The absence of gastric vibrational therapy frameworks that can transform physiological monitoring into personalized therapeutic intervention with developing contextual awareness and adaptive learning represents a significant gap in current technology offerings.

Population-based algorithms in metabolic health applications present personalization concerns despite the potential for creating standardized intervention protocols. Current systems utilizing fixed dietary guidelines apply identical recommendations to all users regardless of individual sensitivity profiles, failing to recognize that individuals show dramatically different responses where some users are highly sleep-sensitive, others stress-reactive, and others exhibit unique glucose response patterns. The combination of population averaging with independent factor modeling and lack of synergistic interaction discovery presents challenges for creating effective personalized systems that can identify when combinations of factors produce effects exceeding simple additive predictions.

The growing prevalence of obesity and type 2 diabetes affecting over 100 million adults with obesity, 37 million with diabetes, and 96 million with prediabetes in the United States alone has created urgent demand for accessible therapeutic solutions and intuitive intervention interfaces. However, current implementations lack multi-modal integration frameworks that could enable gastric vibrational therapy devices to function as adaptive therapeutic systems with physiological responsiveness, food-aware targeting, and coordinated behavioral monitoring. The absence of controllers capable of processing multiple data streams including chewing cadence, glucose levels, heart rate variability, and food properties limits the potential for creating satiety enhancement systems that transcend traditional passive feedback approaches and establish closed-loop therapeutic intervention capable of adapting to individual user needs.

Therefore, there exists a need for improved systems and methods for adaptive gastric satiety therapy that can detect eating behavior through chewing monitoring, process physiological data to determine target eating parameters, and enable personalized therapeutic intervention through closed-loop control of gastric vibrational therapy devices.

### SUMMARY OF THE INVENTION

It is an object of the present invention to provide a satiety enhancement system for adaptive control of gastric satiety therapy.

It is another object of the present invention to provide a satiety enhancement system comprising a gastric vibrational therapy device configured to deliver non-invasive mechanical stimulation to a gastric region of a user.

It is another object of the present invention to provide a chewing detection sensor configured to monitor eating behavior of the user and generate chewing data indicative of a chewing cadence, a physiological monitoring device configured to acquire physiological data from the user, and a controller in communication with a gastric vibrational therapy device, the chewing detection sensor, and the physiological monitoring device.

It is another object of the present invention to provide a controller configured to receive the chewing data from the chewing detection sensor, receive the physiological data from the physiological monitoring device, determine a target chewing cadence based on the physiological data, calculate a deviation between the chewing cadence and the target chewing cadence, and modulate operational parameters of the gastric vibrational therapy device based on the calculated deviation.

It is another object of the present invention to provide a physiological monitoring device comprising a continuous glucose monitor configured to measure glucose levels of the user and a heart rate variability monitor configured to measure heart rate variability metrics indicative of autonomic nervous system state.

It is another object of the present invention to provide operational parameters of the gastric vibrational therapy device comprising at least one of vibration frequency, vibration amplitude, duty cycle, or stimulation duration, wherein the controller is configured to increase the vibration amplitude when the deviation indicates the chewing cadence exceeds the target chewing cadence.

It is another object of the present invention to provide an image capture device configured to acquire images of food and an image analysis module configured to determine food properties from the acquired images, wherein the food properties comprise at least one of food texture classification, estimated caloric content, macronutrient composition, or glycemic index, and wherein the controller is further configured to determine the target chewing cadence based on the food properties.

It is another object of the present invention to provide that the controller is further configured to receive behavioral data including at least one of meal timing patterns, eating duration, compliance history with target chewing cadence, or social context indicators, and to determine the target chewing cadence further based on the behavioral data.

It is another object of the present invention to provide that the physiological monitoring device may further comprise at least one of a skin temperature sensor configured to measure skin temperature indicative of autonomic nervous system state, an abdominal distention sensor configured to measure changes in abdominal circumference indicative of gastric filling, or a gut peptide sensor configured to measure levels of at least one of glucagon-like peptide-1 (GLP-1), peptide YY (PYY), cholecystokinin (CCK), ghrelin, or leptin.

It is another object of the present invention to provide a safety manager configured to detect vehicle operation and automatically suspend AI-controlled operation of the gastric vibrational therapy device during detected vehicle operation.

It is another object of the present invention to provide that the controller is configured to detect an anticipatory glucose decline pattern from physiological data and to preemptively activate the gastric vibrational therapy device to initiate pre-meal priming before the user begins eating.

It is another object of the present invention to provide that the controller is further configured to receive manually entered gut peptide data and incorporate the manually entered gut peptide data into the determination of the target chewing cadence.

In order to overcome the limitations stated herein, the present invention provides a satiety enhancement system for adaptive control of gastric satiety therapy, a method for adaptive control of gastric satiety therapy, and an adaptive therapy system for gastric satiety therapy. The satiety enhancement system comprises a gastric vibrational therapy device configured to deliver non-invasive mechanical stimulation to a gastric region of a user, a chewing detection sensor configured to monitor eating behavior of the user and generate chewing data indicative of a chewing cadence, a physiological monitoring device configured to acquire physiological data from the user, and a controller in communication with the gastric vibrational therapy device, the chewing detection sensor, and the physiological monitoring device. The controller is configured to receive the chewing data from the chewing detection sensor, receive the physiological data from the physiological monitoring device, determine a target chewing cadence based on the physiological data, calculate a deviation between the chewing cadence and the target chewing cadence, and modulate operational parameters of the gastric vibrational therapy device based on the calculated deviation.

In one aspect, the adaptive therapy system comprises a satiety enhancement system including a gastric vibrational therapy device, a chewing detection sensor, and a physiological monitoring device, an electronic device in communication with the satiety enhancement system configured to receive the chewing data and physiological data, determine a target chewing cadence, calculate a deviation, and generate a control signal to modulate operational parameters of the gastric vibrational therapy device, and a server in communication with the electronic device configured to store historical records including meal events and physiological state data, execute machine learning models using the historical records to generate predictions, and transmit personalized therapy protocols to the electronic device for implementation by the satiety enhancement system.

In one aspect, a multi-position gastric vibrational therapy system comprises a first gastric vibrational therapy device configured to be positioned over a cardio region of a stomach of a user, a second gastric vibrational therapy device configured to be positioned over a pylorus region of the stomach of the user, and a controller in communication with the first gastric vibrational therapy device and the second gastric vibrational therapy device. The controller is configured to independently control a first operating frequency of the first gastric vibrational therapy device and a second operating frequency of the second gastric vibrational therapy device based on differential tissue attenuation between the cardio region and the pylorus region to deliver a target stimulation frequency in the range of 60-100 Hz to nerve fibers at each region, wherein the first operating frequency and the second operating frequency are each in the range of 80-250 Hz.

In another aspect, the multi-position gastric vibrational therapy system further comprises a tissue composition sensor configured to measure subcutaneous tissue thickness at the cardio region and at the pylorus region. The controller is further configured to receive tissue composition data, calculate attenuation factors for each region based on the measured subcutaneous tissue thickness, and determine the operating frequencies based on the attenuation factors to compensate for tissue attenuation and deliver the target stimulation frequency to each region.

In one aspect, the method for adaptive control of gastric satiety therapy comprises delivering, by a gastric vibrational therapy device, non-invasive mechanical stimulation to a gastric region of a user, monitoring, by a chewing detection sensor, eating behavior of the user to generate chewing data indicative of a chewing cadence, acquiring, by a physiological monitoring device, physiological data from the user, receiving, by a controller, the chewing data from the chewing detection sensor, receiving, by the controller, the physiological data from the physiological monitoring device, determining, by the controller, a target chewing cadence based on the physiological data, calculating, by the controller, a deviation between the chewing cadence and the target chewing cadence, and modulating, by the controller, operational parameters of the gastric vibrational therapy device based on the calculated deviation.

In another aspect, monitoring eating behavior comprises processing acoustic signals from the chewing detection sensor to identify chewing events using band-pass filtering and peak detection. Acquiring physiological data comprises receiving continuous glucose monitoring data including a glucose level and a glucose rate-of-change metric. Determining the target chewing cadence comprises reducing the target chewing cadence when the glucose rate-of-change metric exceeds a threshold.

In yet another aspect, the method further comprises acquiring images of food using an image capture device, analyzing the acquired images to determine food properties including a food texture classification, and adjusting the target chewing cadence based on the food texture classification. Adjusting the target chewing cadence comprises decreasing the target chewing cadence when the food texture classification indicates a soft food texture to compensate for reduced mechanical satiety stimulation.

In another aspect, the electronic device is further configured to receive image data from an image capture device and determine food properties from the image data, wherein the target chewing cadence is further determined based on the food properties. The food properties comprise a food texture classification, and the electronic device is further configured to decrease the target chewing cadence when the food texture classification indicates a soft food texture.

In one advantageous feature of the present invention, the satiety enhancement system provides closed-loop control wherein chewing behavior and physiological state are monitored and used to adaptively modulate gastric vibrational therapy in real-time during an eating episode, enabling personalized therapeutic intervention that responds to individual eating patterns and metabolic conditions.

In another advantageous feature of the present invention, the controller is configured to calculate a glucose-based adjustment factor based on the glucose levels and apply the glucose-based adjustment factor to the operational parameters of the gastric vibrational therapy device, thereby providing enhanced therapeutic intervention when elevated glucose levels or concerning glucose trends are detected.

In another advantageous feature of the present invention, the controller is configured to generate a risk score based on the physiological data and modulate the operational parameters of the gastric vibrational therapy device based on the risk score, enabling proactive therapeutic intervention based on comprehensive assessment of metabolic state.

In another advantageous feature of the present invention, the gastric vibrational therapy device delivers non-invasive mechanical stimulation to activate vagal nerve fibers and promote satiety, providing therapeutic benefit through physiological mechanisms that do not depend entirely on user behavioral compliance with eating pace guidance.

In another advantageous feature of the present invention, the image analysis module determines food texture classification and the controller decreases the target chewing cadence when soft food texture is detected, thereby compensating for reduced mechanical satiety stimulation by encouraging slower eating that provides additional time for satiety signals to develop.

### BRIEF DESCRIPTION OF FIGURES

FIG. 1 illustrates a block diagram of a system for adaptive control of gastric satiety therapy, in accordance with one embodiment of the present invention.

FIG. 2 illustrates a workflow for promoting effective behavioral modification utilizing satiety induced by gastric stimulation, in accordance with one embodiment of the present invention.

FIG. 3 illustrates a block diagram of an adaptive therapy system comprising a satiety enhancement system, in accordance with one embodiment of the present invention.

FIG. 4 illustrates a block diagram of the satiety enhancement system and internal components thereof, in accordance with one embodiment of the present invention.

FIG. 5 illustrates a block diagram of internal components of a gastric vibrational therapy device, in accordance with one embodiment of the present invention.

FIG. 6 illustrates a method for adaptive control of gastric satiety therapy, in accordance with one embodiment of the present invention.

FIG. 7 illustrates a method for adaptive control of gastric satiety therapy with food image analysis and glucose monitoring, in accordance with one embodiment of the present invention.

FIG. 8 illustrates a method for adaptive control of gastric satiety therapy, in accordance with one embodiment of the present invention.

FIG. 9 illustrates a method for adaptive control of gastric satiety therapy with food texture-based target chewing cadence adjustment, in accordance with one embodiment of the present invention.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

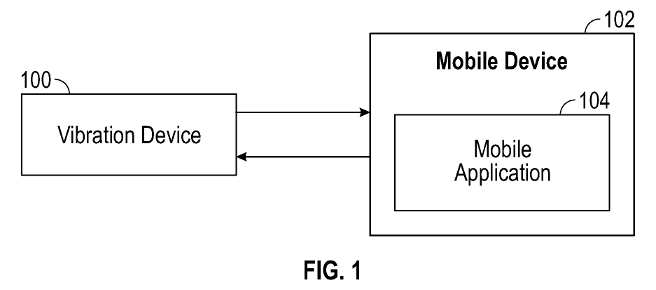

Referring to FIG. 1, a system for adaptive control of gastric satiety therapy is shown, in accordance with one exemplary embodiment of the present invention. The system may comprise a vibration device 100 and a mobile device 102 having a mobile application 104. The vibration device 100 may be an external vibrational device configured to deliver non-invasive mechanical stimulation to a gastric region of a user to activate vagal nerve fibers and promote satiety by stimulating the stomach externally. The vibration device 100 may feature customizable frequency and intensity settings, allowing personalized therapy based on individual physiological needs. The mobile device 102 may be a smartphone, tablet, or other portable computing device configured to host the mobile application 104. The mobile application 104 may serve as an artificial intelligence (AI)-driven companion application for the vibration device 100, providing processing functions for controlling both chewing feedback and gastric vibrational therapy.

The vibration device 100 may communicate wirelessly with the mobile application 104 running on the mobile device 102, enabling seamless data transfer and real-time interaction between the components. The wireless communication may be bidirectional, enabling data transfer from the vibration device 100 to the mobile application 104 and control signals from the mobile application 104 to the vibration device 100. The bidirectional communication may facilitate real-time tracking, therapy optimization, and compliance monitoring. The wireless communication may be implemented using Bluetooth, WiFi, or other wireless communication protocols.

With continued reference to FIG. 1, the mobile application 104 may process physiological and behavioral data to provide personalized recommendations for vibration therapy and lifestyle modifications. The physiological and behavioral data may include eating habits, physical activity, sleep patterns, stress levels, and hormonal fluctuations. The mobile application 104 may receive physiological data from external sensors and monitoring devices, process the received data, and generate control signals for the vibration device 100 based on the processed data. The mobile application 104 may continuously learn from user data to provide personalized recommendations, track trends in weight, dietary habits, and activity, while offering real-time feedback and educational content tailored to user queries.

The mobile application 104 may serve as a controller implemented on the mobile device 102, functioning as a processing component that controls both chewing feedback and gastric vibrational therapy. The mobile application 104 may determine target chewing cadence based on physiological data, calculate deviations between actual chewing cadence and target chewing cadence, and modulate operational parameters of the vibration device 100 based on the calculated deviations. The mobile application 104 may prompt users to utilize the vibration device 100 based on AI-driven analytics that determine optimal timing and duration of therapy. The vibration device 100 may be turned on or adjusted manually, through AI/app notification, or directly by AI.

The synchronization between the vibration device 100 and the mobile application 104 may enable users to receive therapy tailored to individual physiological conditions and eating behaviors, enhancing the overall effectiveness of the weight management program. The mobile application 104 may provide real-time feedback on therapy sessions, monitor compliance with therapy protocols, and offer personalized recommendations based on collected data. The interface between the vibration device 100 and the mobile application 104 may promote user engagement through interactive features and progress tracking. To enhance personalization, the vibration device 100 may integrate with health monitoring systems such as continuous glucose monitors (CGMs) and smartwatches, offering seamless data collection for a weight management solution. The vibration device 100 may dynamically adjust its vibration patterns based on real-time user data, improving therapy efficacy.

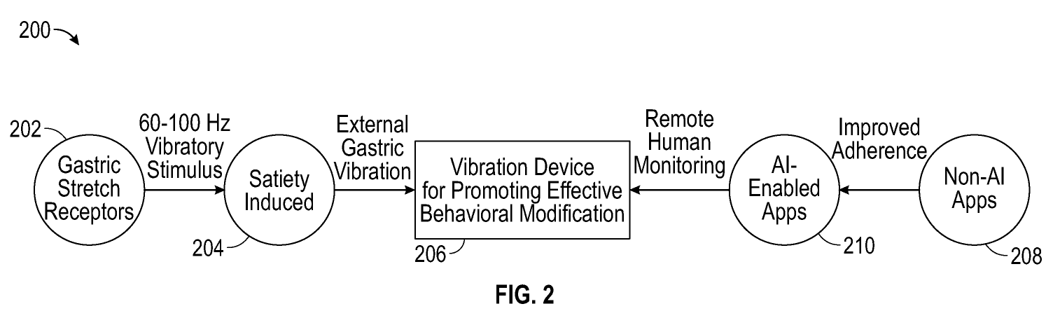

Referring to FIG. 2, a workflow 200 for promoting effective behavioral modification utilizing satiety induced by gastric stimulation is shown, in accordance with one exemplary embodiment of the present invention. The workflow 200 illustrates a sequence of stages and pathways through which gastric vibrational therapy may promote behavioral modification and improved adherence to weight management protocols. The process may begin with AI-driven analytics, which assess the user's physiological and behavioral data to determine the optimal timing and settings for the vibrational device.

The workflow 200 begins with gastric stretch receptors 202 receiving a vibratory stimulus. The gastric stretch receptors 202 may receive a vibratory stimulus in a frequency range of 60-100 Hz from an external gastric vibration source. The vibratory stimulus applied to the gastric stretch receptors 202 may activate vagal nerve fibers and initiate satiety signaling pathways. The gastric stretch receptors 202 may respond to the mechanical stimulation by generating afferent signals that propagate through vagal pathways to brain regions involved in appetite regulation.

With continued reference to FIG. 2, the gastric stretch receptors 202 connect to a satiety induced 204 stage through external gastric vibration. The satiety induced 204 stage represents the physiological response to stimulation of the gastric stretch receptors 202, wherein the user may experience enhanced feelings of fullness and reduced appetite. The satiety induced 204 stage may involve release of satiety hormones, activation of vagal afferent signaling, and modulation of appetite-regulating neural circuits.

The satiety induced 204 stage leads to a vibration device for promoting effective behavioral modification 206. The vibration device for promoting effective behavioral modification 206 represents the therapeutic intervention component that delivers the vibratory stimulus to the gastric stretch receptors 202 and facilitates behavioral changes through the induced satiety response. The vibration device for promoting effective behavioral modification 206 may operate in coordination with monitoring and application components to provide a behavioral modification approach. The mobile application 104 may then prompt the user to initiate the therapy session, providing motivational challenges and tracking progress.

As further shown in FIG. 2, the vibration device for promoting effective behavioral modification 206 may be used in conjunction with three different monitoring and application options within the workflow 200. A first option involves remote human monitoring, enabling healthcare providers to monitor therapy compliance, review data trends, and offer personalized recommendations via a mobile application interface. The remote human monitoring option may provide professional guidance in conjunction with AI-driven recommendations, allowing for adjustments to therapy protocols based on clinical assessments. The integration of human oversight may enhance patient confidence, foster adherence, and ensure safe, effective use of the device.

A second option for use with the vibration device for promoting effective behavioral modification 206 involves an AI-enabled application 210. The AI-enabled application 210 may provide automated analytics, personalized therapy recommendations, and adaptive control of the vibration device for promoting effective behavioral modification 206 based on collected physiological and behavioral data. The AI-enabled application 210 may continuously learn from user data to provide personalized recommendations for vibration therapy and lifestyle modifications. The AI-enabled application 210 may track trends in weight, dietary habits, and activity while offering real-time feedback and educational content tailored to user queries. The AI-enabled application 210 may be compatible with CGMs and smartwatches to enhance its data-driven approach, creating a comprehensive and adaptive weight loss solution.

A third option for use with the vibration device for promoting effective behavioral modification 206 involves a non-AI-enabled application 208. The non-AI-enabled application 208 may provide basic tracking and monitoring without automated analytics or adaptive control features. The non-AI-enabled application 208 may enable users to manually log therapy sessions, track progress, and access educational content. Both the AI-enabled application 210 and the non-AI-enabled application 208 contribute to improved adherence within the workflow 200 by providing users with tools for monitoring and managing therapy protocols. The continuous feedback loop between the device, app, and user fosters long-term adherence to the weight management program, ensuring sustainable results.

The workflow 200 may incorporate virtual reality (VR) modules addressing emotional eating. The integration of virtual reality experiences aims to combat mood-related eating disorders by reinforcing positive dietary behaviors and enhancing satiety cues. The VR modules may target emotional eating behaviors by pairing gastric stimulation with VR scenarios to recondition responses to unhealthy foods. The AI-enabled application 210 may identify correlations between emotional states and eating behaviors using a mood tracker. For instance, users experiencing sadness and sugar cravings may be guided to calming VR scenarios, such as swimming with dolphins, paired with protein-rich meals to promote satiety and positive emotions. Conversely, aversive VR therapy may recondition responses to unhealthy foods by pairing gastric stimulation with unpleasant VR scenarios, discouraging addictive eating habits. These interventions may be evidence-based and designed to support emotional regulation and sustainable behavior changes.

The workflow 200 may include a chatbot feature enabling users to seek guidance and clarification on optimal weight loss strategies, thus fostering sustained adherence. The chatbot feature may be implemented within the AI-enabled application 210 or the non-AI-enabled application 208, allowing users to obtain clarification on therapy protocols, dietary recommendations, and lifestyle modifications. The chatbot feature may foster sustained adherence by providing accessible guidance and support throughout the behavioral modification process. Users may be prompted to participate in motivational challenges to encourage device use and diet/exercise compliance.

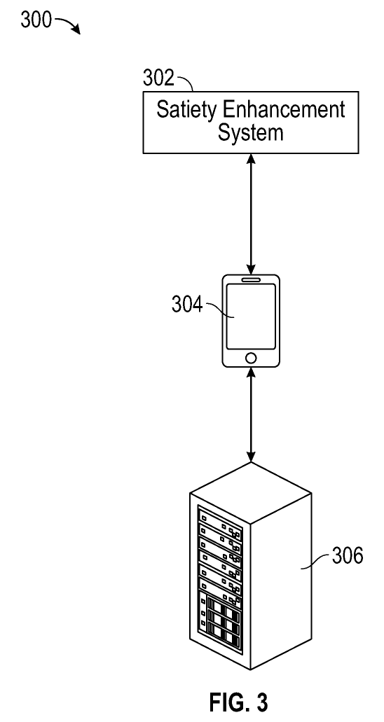

Referring to FIG. 3, an adaptive therapy system 300 is shown, in accordance with one exemplary embodiment of the present disclosure. The adaptive therapy system 300 may comprise a satiety enhancement system 302, an electronic device 304, and a server 306. The adaptive therapy system 300 may provide a distributed architecture for delivering therapeutic intervention for appetite control and metabolic modulation through coordination of sensing, processing, and actuation components across multiple interconnected devices.

The satiety enhancement system 302 may be configured to deliver therapeutic intervention for appetite control and metabolic modulation. The satiety enhancement system 302 may include sensing components for detecting eating behavior and acquiring physiological data, processing components for analyzing the detected behavior and acquired data, and actuation components for delivering non-invasive mechanical stimulation to a gastric region of a user. The satiety enhancement system 302 may operate in coordination with the electronic device 304 and the server 306 to provide adaptive gastric satiety therapy.

With continued reference to FIG. 3, the electronic device 304 may be in communication with the satiety enhancement system 302. The electronic device 304 may be a smartphone, tablet, or other portable computing device configured to serve as an interface for user interaction and data processing. The electronic device 304 may host software applications configured to receive physiological data from the satiety enhancement system 302, process chewing detection information, and generate control signals for the satiety enhancement system 302. The electronic device 304 may communicate bidirectionally with the satiety enhancement system 302, enabling real-time data transfer from the satiety enhancement system 302 to the electronic device 304 and transmission of control signals from the electronic device 304 to the satiety enhancement system 302. The bidirectional communication between the satiety enhancement system 302 and the electronic device 304 may enable therapy optimization based on detected eating behavior and physiological state.

The electronic device 304 may be further in communication with the server 306. The server 306 may provide cloud-based computing resources for data storage, machine learning model execution, and remote monitoring functions. The server 306 may store historical records including meal events, physiological state data, temporal and behavioral context, predictions made by machine learning models, protocols delivered, and actual outcomes observed. The stored historical records may enable machine learning model training and pattern discovery for personalized therapy protocols.

As further shown in FIG. 3, the bidirectional communication between the electronic device 304 and the server 306 may facilitate synchronization of user data, retrieval of personalized therapy protocols, and continuous learning from historical meal data. The electronic device 304 may transmit data collected from the satiety enhancement system 302 to the server 306 for storage and processing. The server 306 may execute machine learning models using the transmitted data to generate predictions, discover individual sensitivity profiles, and determine therapeutic protocols suited to individual users. The predictions generated by the server 306 may include predicted glucose responses to specific foods, predicted eating behaviors and compliance likelihood, predicted postprandial glucose excursions, and predicted satiety outcomes based on current physiological state and food properties. The server 306 may execute computationally intensive machine learning models including gradient boosting models and multi-stream neural networks that benefit from cloud-based processing resources, while the electronic device 304 may handle real-time processing tasks including chewing cadence calculation, deviation computation, and control signal generation that require low-latency response. The server 306 may transmit the generated predictions and determined protocols back to the electronic device 304 for implementation by the satiety enhancement system 302.

The interconnection between the satiety enhancement system 302, the electronic device 304, and the server 306 may enable a distributed architecture where sensor data acquisition, local processing, and cloud-based analytics operate in coordination to provide adaptive gastric satiety therapy. The satiety enhancement system 302 may acquire sensor data and deliver therapeutic stimulation. The electronic device 304 may provide local processing functions for real-time control and user interface functions. The server 306 may provide cloud-based processing functions for machine learning model execution and long-term data storage. The distributed architecture may enable the adaptive therapy system 300 to leverage both local processing for real-time responsiveness and cloud-based processing for computationally intensive machine learning operations.

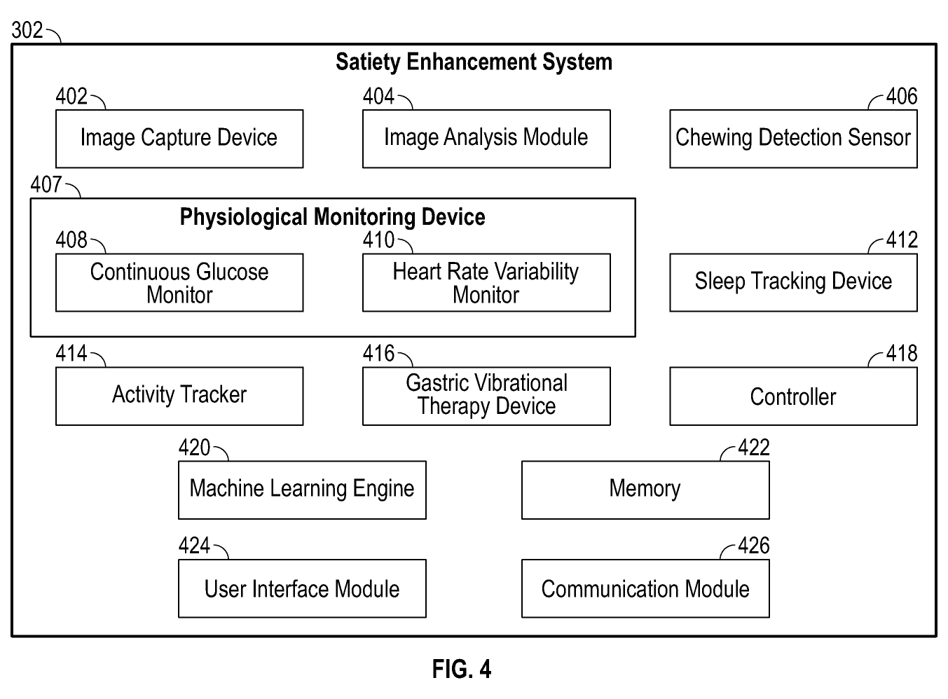

Referring to FIG. 4, a satiety enhancement system 302 and internal components of the satiety enhancement system 302 are shown, in accordance with one exemplary embodiment of the present disclosure. The satiety enhancement system 302 may be configured to provide adaptive control of gastric satiety therapy through integration of multiple sensing modalities, data processing, and therapeutic intervention. The satiety enhancement system 302 may include sensing components, processing components, and actuation components that operate in coordination to detect eating behavior, acquire physiological data, and deliver therapeutic stimulation based on the detected behavior and acquired data.

The satiety enhancement system 302 may include an image capture device 402 configured to acquire images of food before consumption. The image capture device 402 may be a camera integrated into a smartphone, tablet, dedicated device, or wearable device such as smart glasses. The image capture device 402 may be in communication with an image analysis module 404. The image analysis module 404 may be configured to process food images acquired by the image capture device 402 to extract food properties. The food properties may include food identification, texture classification, portion size estimation, macronutrient composition, caloric content, and glycemic index prediction. The image analysis module 404 may employ convolutional neural networks trained on food image databases to identify foods present in acquired images and determine texture characteristics of the identified foods.

With continued reference to FIG. 4, the satiety enhancement system 302 may include a chewing detection sensor 406 configured to monitor eating behavior of a user and generate chewing data indicative of a chewing cadence. The chewing detection sensor 406 may generate chewing data indicative of chewing cadence, bite frequency, and swallow events during an eating episode. The chewing detection sensor 406 may be positioned on or near the user's head, jaw, neck, or ear region to detect eating behavior. In some cases, the chewing detection sensor 406 may comprise an ear-mounted sensor, a temple-mounted sensor, a jaw-mounted sensor, or a neck-mounted sensor.

The chewing detection sensor 406 may comprise various sensor types configured to detect eating behavior through different sensing modalities. The chewing detection sensor 406 may comprise at least one of an acoustic sensor, an inertial measurement unit, an electromyographic sensor, or a strain sensor. Each sensor type may detect chewing activity through a distinct physical mechanism, and the selection of sensor type may depend on user preferences, wearing comfort, and detection accuracy requirements for a given application.

The satiety enhancement system 302 may include multiple physiological monitoring devices configured to acquire physiological data from the user. The continuous glucose monitor 408 may be configured to measure interstitial glucose levels of the user continuously. The continuous glucose monitor 408 may provide real-time glucose data including current glucose level, trend direction indicating whether glucose is rising, falling, or stable, and rate of change of glucose levels. The continuous glucose monitor 408 may comprise a wearable device with a subcutaneous sensor that measures glucose levels at regular intervals.

The controller 418 may detect an anticipatory glucose decline pattern indicative of impending food consumption. Glucose levels may begin to decline approximately 15 to 30 minutes before eating commences, representing a physiological anticipatory response to expected food intake. The controller 418 may monitor glucose data from the continuous glucose monitor 408 to detect a falling glucose trend that matches the characteristics of an anticipatory glucose decline. When an anticipatory glucose decline is detected, the controller 418 may preemptively activate the gastric vibrational therapy device 416 to initiate pre-meal priming before the user begins eating. The preemptive activation based on anticipatory glucose decline may enable the system to begin vagal activation and satiety pathway priming before food consumption commences, potentially enhancing the effectiveness of the therapeutic intervention. The controller 418 may distinguish anticipatory glucose decline from other causes of falling glucose, such as insulin-mediated glucose uptake following a previous meal, based on the timing, magnitude, and pattern of the glucose decline in conjunction with contextual factors such as time since last meal and typical meal timing patterns for the user.

The satiety enhancement system 302 may include a heart rate variability monitor 410. The heart rate variability monitor 410 may be configured to measure beat-to-beat heart rate intervals of the user. The heart rate variability monitor 410 may calculate heart rate variability metrics indicative of autonomic nervous system state and vagal tone. The heart rate variability metrics may include time domain metrics such as root mean square of successive differences (RMSSD) and standard deviation of normal-to-normal intervals (SDNN), and frequency domain metrics such as low frequency power, high frequency power, and low frequency to high frequency ratio. The heart rate variability monitor 410 may comprise a chest strap, a wrist-worn optical sensor, or a finger pulse oximeter.

The satiety enhancement system 302 may include a sleep tracking device 412. The sleep tracking device 412 may be configured to monitor sleep duration, quality, and architecture of the user. The sleep tracking device 412 may comprise a wearable tracker, an under-mattress sensor, a bedside radar-based monitor, or a smartphone application. The sleep tracking device 412 may provide data including total sleep time, sleep efficiency, percentage of time in each sleep stage including light sleep, deep sleep, and rapid eye movement (REM) sleep, number of awakenings, and overall sleep quality scores.

The satiety enhancement system 302 may include an activity tracker 414. The activity tracker 414 may be configured to monitor physical activity of the user. The activity tracker 414 may monitor step count, active minutes, exercise sessions, sedentary time, and energy expenditure. The activity tracker 414 may provide context on recent exercise timing and intensity affecting metabolic state of the user. The activity tracker 414 may be integrated with wearable fitness trackers or implemented as a standalone device.

The physiological monitoring device 407 may further comprise a skin temperature sensor configured to measure skin temperature of the user. Skin temperature may provide an indicator of peripheral blood flow and autonomic nervous system state. Changes in skin temperature may correlate with stress responses, circadian rhythm phase, and metabolic activity. The skin temperature sensor may be integrated into a wearable device positioned on the wrist, finger, or other body location. The controller 418 may receive skin temperature data from the skin temperature sensor and may incorporate the skin temperature data into the determination of target chewing cadence and therapeutic protocol parameters. A decrease in peripheral skin temperature may indicate sympathetic activation associated with stress, while an increase in peripheral skin temperature may indicate parasympathetic activation associated with relaxation and favorable digestive conditions.

The physiological monitoring device 407 may further comprise an abdominal distention sensor configured to measure changes in abdominal circumference or pressure indicative of gastric filling. The abdominal distention sensor may comprise a strain gauge, a pressure sensor, or an impedance sensor positioned on an abdominal belt or integrated into a wearable garment. The abdominal distention sensor may detect increases in abdominal circumference or pressure that occur as food enters the stomach during an eating episode. The controller 418 may receive abdominal distention data from the abdominal distention sensor and may use the abdominal distention data to assess gastric filling status and adjust therapeutic intervention accordingly. Rapid increases in abdominal distention may indicate rapid food consumption, and the controller 418 may increase the intensity of gastric vibrational therapy in response to detected rapid gastric filling.

The satiety enhancement system 302 may include additional sensors and tracking modules. The satiety enhancement system 302 may include galvanic skin response sensors for stress detection. The satiety enhancement system 302 may include menstrual cycle tracking via calendar-based or temperature-based methods. The satiety enhancement system 302 may include medication tracking modules for tracking active medications affecting metabolic state. The satiety enhancement system 302 may include location/GPS sensors for detecting social and environmental context. The satiety enhancement system 302 may include ambient environment sensors measuring noise and lighting conditions.

The physiological monitoring device 407 may further comprise a gut peptide sensor configured to measure levels of gut peptides in the user. Gut peptides are hormones secreted by the gastrointestinal tract that play a role in appetite regulation and satiety signaling. The gut peptides may include glucagon-like peptide-1 (GLP-1), peptide YY (PYY), cholecystokinin (CCK), ghrelin, and leptin. GLP-1 and PYY are satiety hormones that increase following food consumption and promote feelings of fullness. CCK is released in response to fat and protein consumption and promotes satiety. Ghrelin is a hunger hormone that increases before meals and decreases after eating. Leptin is an adiposity signal that reflects long-term energy stores. The gut peptide sensor may comprise a wearable biosensor configured to measure gut peptide levels through interstitial fluid sampling, microneedle-based sensing, or other minimally invasive or non-invasive sensing modalities. The controller 418 may receive gut peptide data from the gut peptide sensor and may incorporate the gut peptide data into the determination of target chewing cadence, risk score calculation, and therapeutic protocol parameters.

In some embodiments, gut peptide data may be received through manual input by the user or a healthcare provider. The user interface module 424 may provide an input interface enabling the user to enter gut peptide measurement values obtained from laboratory testing or point-of-care testing devices. The manually entered gut peptide data may include GLP-1 levels, ghrelin levels, leptin levels, or other gut peptide measurements. The controller 418 may incorporate the manually entered gut peptide data into the machine learning models and therapeutic protocol determination. The manual input of gut peptide data may enable the system to account for hormonal factors affecting appetite and satiety even when continuous gut peptide sensing is not available. The manually entered gut peptide data may be timestamped and stored in the memory 422 for use in longitudinal analysis of hormonal patterns and their relationship to eating behavior and metabolic outcomes.

The satiety enhancement system 302 may include a gastric vibrational therapy device 416 configured to deliver non-invasive mechanical stimulation to a gastric region of the user. The gastric vibrational therapy device 416 may apply controlled mechanical vibration to stimulate gastric mechanoreceptors, enhance vagal signaling, promote satiety hormone secretion, and influence gastric emptying. The gastric vibrational therapy device 416 may be positioned on an abdominal region of the user over the gastric fundus or body region.

The satiety enhancement system 302 may include a controller 418. The controller 418 may be in communication with the image capture device 402, the image analysis module 404, the chewing detection sensor 406, the continuous glucose monitor 408, the heart rate variability monitor 410, the sleep tracking device 412, the activity tracker 414, and the gastric vibrational therapy device 416. The controller 418 may be configured to receive data from the various sensors and monitoring devices, determine target chewing cadence based on the received data, calculate deviations between actual chewing cadence and target chewing cadence, and modulate operational parameters of the gastric vibrational therapy device 416 based on the calculated deviations. The controller 418 may comprise a processor, a baseline protocol determination module, a real-time modulation module, a compensatory protocol module, and a prediction module.

The system may include two or more gastric vibrational therapy devices 416 positioned at different locations on the stomach to target different nerve areas. The gastric vibrational therapy devices 416 may be positioned at a cardio region at the top of the stomach and at a pylorus region at the bottom of the stomach. The cardio region and the pylorus region contain different populations of nerve fibers involved in satiety signaling. By positioning gastric vibrational therapy devices 416 at both the cardio region and the pylorus region, the system may stimulate nerve fibers at multiple anatomical locations to enhance overall satiety response.

The nerves that signal fullness are located at the top of the stomach and at the bottom of the stomach. Studies have mapped the distribution of vagal afferent fibers in the gastric wall, demonstrating that mechanoreceptors and nerve endings are concentrated in both the fundus/cardio region and the antrum/pylorus region. The gastric vibrational therapy devices 416 positioned at these different locations may activate distinct populations of vagal afferent fibers, providing complementary stimulation pathways for satiety signaling.

Transmission of mechanical vibration over distance through tissue creates attenuation of the vibrational signal. The distance from the skin surface to the pylorus region at the bottom of the stomach may be shorter than the distance from the skin surface to the cardio region at the top of the stomach. When a single gastric vibrational therapy device 416 is positioned at one location, the mechanical vibration may reach the nerve fibers at that location with a first stimulation strength while reaching nerve fibers at a more distant location with a reduced stimulation strength due to attenuation. For example, a gastric vibrational therapy device 416 positioned over the pylorus region and operating at a frequency of 200 Hz may deliver stimulation at 80 Hz to nerve fibers at the pylorus region while delivering stimulation at only 50 Hz to nerve fibers at the more distant cardio region due to attenuation over the greater transmission distance.

Positioning gastric vibrational therapy devices 416 at each location may enable the system to achieve a desired stimulation strength at both the cardio region and the pylorus region. Each gastric vibrational therapy device 416 may be configured with operational parameters selected to account for the transmission distance from the skin surface to the target nerve fibers at that location. The gastric vibrational therapy device 416 positioned over the cardio region may operate at parameters selected to deliver a desired stimulation strength to nerve fibers in the cardio region. The gastric vibrational therapy device 416 positioned over the pylorus region may operate at parameters selected to deliver a desired stimulation strength to nerve fibers in the pylorus region. The independent positioning and parameter configuration of each gastric vibrational therapy device 416 may enable the system to achieve consistent stimulation strength across multiple nerve areas despite differences in transmission distance and tissue attenuation.

Each gastric vibrational therapy device 416 may be individually controlled. Individual control may enable each gastric vibrational therapy device 416 to operate with different operational parameters including different vibration frequencies, different vibration amplitudes, different duty cycles, and different stimulation durations. The individual control may enable the system to provide differentiated stimulation to the cardio region and the pylorus region based on the specific nerve populations at each location and the therapeutic objectives for a given eating episode.

In some cases, one gastric vibrational therapy device 416 may be manually controlled while another gastric vibrational therapy device 416 may be AI-controlled. The manually controlled gastric vibrational therapy device 416 may operate at user-selected parameters that remain constant throughout a therapy session. The AI-controlled gastric vibrational therapy device 416 may operate at parameters that are automatically adjusted based on detected eating behavior, physiological data, and calculated deviations from target chewing cadence. The combination of manual control and AI control may enable users to maintain direct control over one aspect of therapy while benefiting from adaptive control for another aspect.

In some cases, both gastric vibrational therapy devices 416 may be AI-controlled. The AI-controlled configuration may enable the system to coordinate operation of both gastric vibrational therapy devices 416 based on comprehensive analysis of eating behavior and physiological state. The system may activate both gastric vibrational therapy devices 416 simultaneously when elevated glucose levels or high-risk eating conditions are detected. The system may activate the gastric vibrational therapy devices 416 sequentially or with different timing patterns based on the progression of an eating episode. The coordinated AI control of multiple gastric vibrational therapy devices 416 may enable the system to provide enhanced therapeutic intervention by stimulating multiple nerve areas in a coordinated manner.

The multiple gastric vibrational therapy device 416 configuration may enable the system to address individual differences in nerve distribution and sensitivity among users. Some users may exhibit greater sensitivity to stimulation at the cardio region while other users may exhibit greater sensitivity to stimulation at the pylorus region. The system may learn individual sensitivity profiles through machine learning analysis of historical meal data and adjust the relative stimulation intensity at each location based on the discovered sensitivity profile. Users who respond more strongly to cardio region stimulation may receive enhanced stimulation at the cardio region, while users who respond more strongly to pylorus region stimulation may receive enhanced stimulation at the pylorus region.

The satiety enhancement system 302 may include a machine learning engine 420 configured to learn from historical meal data. The machine learning engine 420 may predict glucose responses based on current state, predict eating behaviors and compliance likelihood, discover individual sensitivity profiles, identify synergistic interaction effects, and determine therapeutic protocols for current conditions. The machine learning engine 420 may comprise multiple specialized models for glucose prediction, behavior prediction, and protocol optimization.

The satiety enhancement system 302 may include a safety manager configured to inhibit operation of the gastric vibrational therapy device 416 when unsafe conditions are detected. The safety manager may monitor physiological data from the physiological monitoring device 407 to detect conditions that may contraindicate gastric vibrational therapy. Unsafe conditions may include abnormal heart rate exceeding upper or lower thresholds, extreme glucose levels indicating severe hypoglycemia or hyperglycemia, excessive device temperature exceeding safe limits for skin contact, abnormal heart rate variability patterns indicating acute physiological distress, or user-reported discomfort. When an unsafe condition is detected, the safety manager may automatically suspend operation of the gastric vibrational therapy device 416 and generate an alert to the user through the user interface module 424. The safety manager may provide a manual override capability enabling the user to pause or resume therapy operation. The manual override may require user confirmation to resume therapy after an automatic suspension due to detected unsafe conditions.

The safety manager may further be configured to detect when the user is operating a vehicle and automatically suspend AI-controlled operation of the gastric vibrational therapy device 416 and associated audio cues during vehicle operation. The safety manager may detect vehicle operation through one or more detection methods including integration with vehicle systems via Bluetooth, Wi-Fi, or other wireless protocols that indicate connection to a vehicle infotainment system, detection of driving-related motion patterns using accelerometer data from the electronic device 304 or a wearable sensor, GPS-based detection of travel speed exceeding a threshold indicative of vehicle travel, detection of connection to a vehicle charging port, or user input indicating driving status. When vehicle operation is detected, the safety manager may automatically suspend AI-controlled therapeutic intervention to prevent distraction during driving. The safety manager may also suspend or modify audio cues, haptic feedback, and visual notifications that could distract the user during vehicle operation. The safety manager may resume normal AI-controlled operation when vehicle operation is no longer detected. The automatic suspension during vehicle operation may enhance user safety by preventing therapeutic interventions that could distract from the driving task.

The satiety enhancement system 302 may include a memory 422. The memory 422 may be configured to store historical records for the user. The memory 422 may store meal events with associated food properties, physiological state data, temporal and behavioral context, predictions made, protocols delivered, actual outcomes observed, and prediction errors. The memory 422 may enable the machine learning engine 420 to perform model training and pattern discovery based on the stored historical records.

The satiety enhancement system 302 may include a user interface module 424. The user interface module 424 may provide food image capture, meal logging, notifications, recommendations, educational content, progress tracking, and settings management. The user interface module 424 may display current risk assessment, gastric vibrational therapy device status, glucose trends, and personalized insights to the user. The satiety enhancement system 302 may include a communication module 426. The communication module 426 may enable wireless data exchange between components of the satiety enhancement system 302 via Bluetooth, WiFi, cellular, or other wireless communication protocols. The communication module 426 may facilitate synchronization of data across devices and cloud backup of stored data.

The satiety enhancement system 302 may include an audio output device configured to deliver synchronized audio cues to guide chewing behavior of the user. The audio output device may comprise a speaker integrated into an earbud, a smartphone, a smartwatch, or another wearable device. The synchronized audio cues may be configured to induce the user to temporally align mastication events with the target chewing cadence determined by the controller 418. The synchronized audio cues may comprise rhythmic tones, verbal prompts, musical beats, or combinations thereof. Rhythmic tones may provide a steady beat at the target chewing cadence frequency, enabling the user to synchronize chewing with the audible rhythm. Verbal prompts may provide spoken instructions such as "slow down" or "maintain pace" based on detected deviation from the target chewing cadence. Musical beats may provide tempo-guided audio that adjusts dynamically based on the target chewing cadence. The controller 418 may dynamically adapt the timing of the synchronized audio cues based on detected deviation between the target chewing cadence and the user's current chewing rate. When the chewing cadence aligns with the target chewing cadence within a tolerance window, the controller 418 may reduce the intensity or frequency of the audio cues. When the chewing cadence deviates from the target chewing cadence, the controller 418 may increase the intensity or frequency of the audio cues to encourage pace correction.

The satiety enhancement system 302 may be implemented in various form factors. In some embodiments, the satiety enhancement system 302 may be configured as a device attached directly to the user's skin, wherein the sensing components, processing components, and actuation components are integrated into a single wearable unit that adheres to the skin surface using a medical-grade adhesive. In other embodiments, the satiety enhancement system 302 may be implemented as a hybrid configuration, wherein some components such as the chewing detection sensor 406 and the gastric vibrational therapy device 416 are attached to the user's skin while other components such as the controller 418 and the user interface module 424 are implemented on a separate device such as a smartphone or wearable computing device. In yet further embodiments, the satiety enhancement system 302 may be provided as a separate, standalone device configuration, wherein the system comprises multiple discrete devices that communicate wirelessly via the communication module 426, with each device housing specific components of the satiety enhancement system 302.

The acoustic sensor may detect chewing sounds generated during mastication. Chewing produces characteristic sound patterns in a frequency range of 1-4 kHz with rhythmic repetition corresponding to the chewing cadence. The acoustic sensor may comprise a microphone configured to capture audio signals in the frequency range associated with chewing activity. The captured audio signals may be processed using digital signal processing algorithms to identify chewing events and calculate chewing cadence.

The chewing detection sensor 406 may comprise an ear-mounted microphone positioned in an earbud or in-ear monitor for intimate proximity to chewing sounds. The ear-mounted microphone may be integrated into an earbud worn by the user during eating episodes. The positioning of the ear-mounted microphone within the ear canal may provide intimate proximity to chewing sounds generated by jaw movement and food mastication. The intimate proximity may enable the ear-mounted microphone to capture chewing sounds with high signal-to-noise ratio compared to microphones positioned at greater distances from the chewing source. The earbud or in-ear monitor configuration may provide a convenient and unobtrusive form factor for daily use during meals.

The chewing detection sensor 406 may comprise a smart glasses temple-mounted sensor for bone conduction detection of chewing. The smart glasses temple-mounted sensor may be integrated into the temple arm of smart glasses worn by the user. The temple arm of smart glasses may rest against the temporal bone of the skull, which transmits mechanical vibrations generated by jaw movement during chewing. The smart glasses temple-mounted sensor may detect bone-conducted vibrations corresponding to chewing activity. The bone conduction detection mechanism may enable the smart glasses temple-mounted sensor to detect chewing activity even in noisy environments where airborne acoustic signals may be obscured by ambient noise.

The chewing detection sensor 406 may comprise a collar-mounted device for throat-area acoustic monitoring. The collar-mounted device may be positioned on or near the collar region of the user's clothing, placing the device in proximity to the throat area. The throat area may transmit acoustic signals associated with chewing and swallowing activity. The collar-mounted device may detect both chewing sounds and swallowing sounds, enabling the system to monitor multiple aspects of eating behavior. The collar-mounted configuration may provide a convenient attachment location that does not require the user to wear additional head-mounted devices.

The chewing detection sensor 406 may comprise microelectromechanical systems (MEMS) sensors combining accelerometer and gyroscope with a microphone to capture vibrations and sounds. MEMS refers to microelectromechanical systems, which are miniaturized mechanical and electromechanical devices fabricated using microfabrication techniques. The MEMS sensors may include an accelerometer configured to detect linear acceleration associated with jaw movement, a gyroscope configured to detect rotational motion associated with jaw opening and closing, and a microphone configured to detect acoustic signals associated with chewing sounds. The combination of accelerometer, gyroscope, and microphone may enable the chewing detection sensor 406 to capture both mechanical vibrations and acoustic signals generated during eating. The multi-modal sensing approach may improve detection accuracy by enabling the system to correlate signals from multiple sensing modalities to distinguish chewing events from non-chewing activities such as talking or laughing.

The inertial measurement unit may detect jaw movement patterns associated with chewing activity. The inertial measurement unit may comprise an accelerometer and a gyroscope configured to measure acceleration and angular velocity of jaw motion. The inertial measurement unit may be mounted on the head, jaw, or temple region of the user to detect cyclical jaw movements characteristic of chewing. Chewing produces characteristic cyclical jaw movements with specific acceleration signatures that may be distinguished from other head movements through signal processing algorithms.

The electromyographic sensor may detect electrical activity of muscles involved in chewing. The electromyographic sensor may comprise surface electrodes configured to detect electrical signals generated by the masseter muscle and the temporalis muscle during chewing. The masseter muscle and the temporalis muscle are primary muscles involved in jaw closure during mastication. The electromyographic sensor may provide direct measurement of muscle activity associated with chewing, enabling accurate detection of chewing events. The electromyographic sensor may require electrode application to the skin overlying the chewing muscles.

The strain sensor may detect mechanical strain associated with jaw movement during chewing. The strain sensor may comprise piezoelectric or piezoresistive sensing elements configured to detect pressure changes from jaw clenching. The strain sensor may be embedded in an eyeglass temple or an ear-area wearable device to detect mechanical strain transmitted through the skull during jaw closure. The strain sensor may detect the force of jaw closure during each chewing cycle, enabling the system to monitor chewing intensity in addition to chewing cadence.

The chewing detection sensor 406 may be implemented as a wearable patch positioned on the neck or chest area. The wearable patch may comprise a compact housing containing sensing elements, processing electronics, a battery, and a wireless communication module. The wearable patch may be positioned on the upper side of the body, such as on the neck or on the chest, where the wearable patch may capture vibrations and sounds associated with chewing and swallowing activity. The wearable patch may be a battery-operated unit configured to communicate directly with the gastric vibrational therapy device 416 or with a mobile application via a wireless communication protocol such as Bluetooth. The wearable patch may be removable for charging and may be configured to operate for extended periods on a single charge due to the power efficiency of MEMS sensors.

The wearable patch implementation may enable the satiety enhancement system 302 to detect when the user starts eating and to compute eating behavior metrics including chewing rate, swallow frequency, and time intervals between food consumption events. The wearable patch may use proprietary algorithms to compute characteristics of the food being eaten, such as whether the food is hard or soft, based on the vibration and sound patterns detected during chewing. The wearable patch may auto-trigger the gastric vibrational therapy device 416 a specified amount of time after the user starts eating, enabling automated therapy initiation without requiring manual user input.

The gastric vibrational therapy device 416 may attach to an abdomen of a user via multiple attachment mechanisms. A first attachment mechanism may comprise a medical-grade adhesive patch. The medical-grade adhesive patch may be similar to electrocardiogram (ECG) electrodes used for cardiac monitoring applications. The medical-grade adhesive patch may be a single-use or limited reuse adhesive pad that positions the gastric vibrational therapy device 416 over a gastric region of the user. The medical-grade adhesive patch may provide consistent acoustic coupling between the gastric vibrational therapy device 416 and the skin surface of the user, facilitating efficient transmission of mechanical vibration to underlying gastric tissue.

A second attachment mechanism may comprise an adjustable elastic belt worn around a torso of the user. The adjustable elastic belt may be a reusable and washable band that secures the gastric vibrational therapy device 416 at an appropriate position on the abdomen. The gastric vibrational therapy device 416 may clip into the adjustable elastic belt at a designated location corresponding to the gastric region. The adjustable elastic belt may provide less intimate contact with the skin surface compared to the medical-grade adhesive patch but may offer greater convenience for daily wear and repeated use.

A third attachment mechanism may comprise garment integration. The gastric vibrational therapy device 416 may be integrated into or attachable to a compression garment, shirt, or shapewear. The garment-integrated configuration may provide convenient attachment for daily wear by incorporating the gastric vibrational therapy device 416 into clothing worn by the user. The garment-integrated configuration may maintain consistent positioning of the gastric vibrational therapy device 416 relative to the gastric region throughout wear.

The gastric vibrational therapy device 416 may be positioned over a gastric fundus/body region of the user. The positioning may be approximately 2-3 inches left of a midline of the abdomen. The positioning may be approximately 2-4 inches below a xiphoid process of the user. The xiphoid process is a small cartilaginous extension at the bottom of the sternum. The positioning over the gastric fundus/body region may enable the mechanical vibration generated by the gastric vibrational therapy device 416 to reach gastric mechanoreceptors and vagal nerve fibers in the stomach wall.

The positioning of the gastric vibrational therapy device 416 may be adjusted based on body habitus of the user. Body habitus refers to the physical build and body composition of an individual. Users with different body habitus may have different anatomical relationships between external landmarks and the underlying gastric region. The positioning may be adjusted to account for variations in subcutaneous tissue thickness, abdominal wall configuration, and gastric position among different users. A smartphone application may provide positioning guidance with an anatomical diagram and confirmation of placement via a vibration test prompting the user to confirm whether vibration is felt clearly at the intended location.

The satiety enhancement system 302 may include a machine learning engine 420 configured to learn from historical meal data. The machine learning engine 420 may predict glucose responses based on current state, predict eating behaviors and compliance likelihood, discover individual sensitivity profiles, identify synergistic interaction effects, and determine therapeutic protocols for current conditions. The machine learning engine 420 may comprise multiple specialized models for glucose prediction, behavior prediction, and protocol optimization.

With continued reference to FIG. 4, the memory 422 may be configured to store historical records for the user. The memory 422 may store meal events with associated food properties, physiological state data, temporal and behavioral context, predictions made, protocols delivered, actual outcomes observed, and prediction errors. The memory 422 may enable the machine learning engine 420 to perform model training and pattern discovery based on the stored historical records.

The user interface module 424 may provide food image capture, meal logging, notifications, recommendations, educational content, progress tracking, and settings management. The user interface module 424 may display current risk assessment, gastric vibrational therapy device status, glucose trends, and personalized insights to the user. The communication module 426 may enable wireless data exchange between components of the satiety enhancement system 302 via Bluetooth, WiFi, cellular, or other wireless communication protocols. The communication module 426 may facilitate synchronization of data across devices and cloud backup of stored data.

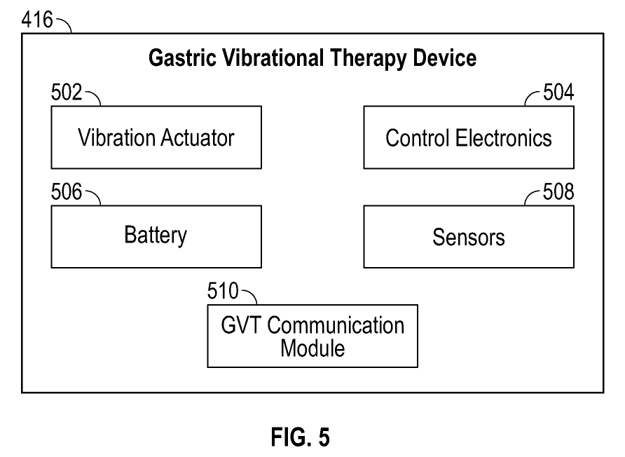

Referring to FIG. 5, a gastric vibrational therapy device 416 and internal components of the gastric vibrational therapy device 416 are shown, in accordance with one exemplary embodiment of the present disclosure. The gastric vibrational therapy device 416 may be configured to deliver non-invasive mechanical stimulation to a gastric region of a user to promote satiety by activating vagal nerve fibers. The gastric vibrational therapy device 416 may comprise a compact housing containing a vibration actuator 502, control electronics 504, a battery 506, sensors 508, and a gastric vibrational therapy (GVT) communication module 510.

The vibration actuator 502 may generate mechanical vibration for therapeutic stimulation of the gastric region. The control electronics 504 may be configured to regulate operation of the vibration actuator 502 and manage operational parameters including vibration frequency, vibration amplitude, and duty cycle. The control electronics 504 may receive control signals through the GVT communication module 510 and generate drive signals for the vibration actuator 502 based on the received control signals.

The battery 506 may provide power to the gastric vibrational therapy device 416. The sensors 508 may include an accelerometer for vibration verification and a temperature sensor for safety monitoring. The accelerometer may verify that the vibration actuator 502 is operating at intended parameters. The temperature sensor may monitor device temperature to ensure the gastric vibrational therapy device 416 remains within acceptable temperature limits for skin contact.

The GVT communication module 510 may enable wireless communication between the gastric vibrational therapy device 416 and external devices such as a mobile application or controller. The GVT communication module 510 may facilitate bidirectional data transfer for real-time therapy optimization and compliance monitoring. The GVT communication module 510 may be implemented using Bluetooth Low Energy (BLE) or other wireless communication protocols. The components within the gastric vibrational therapy device 416 may operate in coordination to deliver controlled mechanical stimulation based on control signals received through the GVT communication module 510.

With continued reference to FIG. 5, the vibration actuator 502 may comprise an eccentric rotating mass (ERM) motor. The ERM motor may be a small direct current (DC) motor with an offset weight that produces circular vibration. The ERM motor may provide a simple and cost-effective mechanism for generating mechanical vibration. In some cases, the vibration actuator 502 may comprise a linear resonant actuator (LRA). The LRA may be a voice coil actuator with a spring-mass system that produces linear vibration. The LRA may provide more precise control of vibration parameters compared to the ERM motor. In some cases, the vibration actuator 502 may comprise a piezoelectric transducer. The piezoelectric transducer may be a crystal-based actuator that provides very precise frequency control and higher frequency capability compared to motor-based actuators.

The control electronics 504 may include a microcontroller, motor driver circuitry, and power management components. The battery 506 may comprise a rechargeable lithium polymer battery. The battery 506 may have a capacity of 500-1000 mAh, providing 8-12 hours of operation per charge. The control electronics 504 may include battery charging circuitry for recharging the battery 506. The compact housing may have dimensions of approximately 8 cm × 5 cm × 2 cm with a weight of 80-120 g.

The gastric vibrational therapy device 416 may include temperature monitoring with automatic shutdown if device temperature exceeds 43 degrees Celsius for skin safety. The gastric vibrational therapy device 416 may include maximum duration limits with automatic shutoff after 3 hours of continuous operation. The gastric vibrational therapy device 416 may include multi-layer acoustic insulation and vibration dampening structures for soundproofing. The acoustic insulation may minimize operational noise generated by the vibration actuator 502 during therapy sessions. The vibration dampening structures may reduce transmission of vibration to the housing exterior, providing discrete operation for user comfort.

The gastric vibrational therapy device 416 may operate with adjustable operational parameters that enable customization of therapeutic stimulation based on user characteristics and detected eating behavior. The operational parameters of the gastric vibrational therapy device 416 may comprise vibration frequency, vibration amplitude, duty cycle, and stimulation duration. These operational parameters may be modulated individually or in combination to provide therapeutic intervention tailored to specific eating episodes and physiological conditions.

Vibration frequency may refer to the rate of vibration oscillation measured in Hertz (Hz). The gastric vibrational therapy device 416 may operate at frequencies in the range of 80-200 Hz. Lower frequencies in the range of 80-120 Hz may provide broader mechanical wave propagation and deeper penetration into gastric tissue. Higher frequencies in the range of 140-200 Hz may provide more localized stimulation and may activate different populations of mechanoreceptors in the gastric wall. The frequency may be selected based on individual user responses and therapeutic objectives for a given eating episode.

Vibration amplitude may refer to the intensity of mechanical displacement measured in g-force. The gastric vibrational therapy device 416 may operate at amplitudes in the range of 0.3-1.5 g-force. Lower amplitudes in the range of 0.3-0.6 g-force may provide gentle stimulation suitable for sensitive users or initial therapy sessions. Higher amplitudes in the range of 0.8-1.5 g-force may provide stronger stimulation for more pronounced therapeutic effect. The amplitude may be adjusted to balance therapeutic efficacy with user comfort and tolerability.

Duty cycle may refer to the percentage of time that vibration is active within a given cycle period. A duty cycle of 100% may indicate continuous vibration throughout the therapy session. A duty cycle of 50% may indicate a pulsed pattern where vibration is active for half of each cycle period and inactive for the remaining half. Variable duty cycles may enable complex patterns where the ratio of active to inactive time changes throughout the therapy session.

Stimulation duration may refer to the total time of gastric vibrational therapy application during a therapy session. Pre-meal stimulation duration may range from 0-15 minutes. Intra-meal stimulation duration may correspond to the duration of eating, which may range from 10-30 minutes. Post-meal stimulation duration may range from 20-120 minutes depending on therapeutic objectives and detected eating behavior.

The gastric vibrational therapy device 416 may operate with vibration patterns including continuous, pulsed, ramped, and complex frequency or amplitude modulation patterns. A continuous pattern may provide constant vibration at a set frequency and amplitude throughout the therapy session. A pulsed pattern may provide on/off cycling at a specified pulse rate, such as 0.5 seconds on and 0.5 seconds off at a 1 Hz pulse rate. A ramped pattern may provide gradual increase or decrease in amplitude over time, such as a ramped-down pattern that gradually decreases amplitude throughout a post-meal therapy session. A complex pattern may provide frequency or amplitude modulation following a programmed profile, enabling sophisticated stimulation sequences that vary multiple parameters throughout the therapy session.

The operational parameters may be modulated based on calculated deviations between actual chewing cadence and target chewing cadence. When the controller 418 calculates a deviation indicating that the chewing cadence exceeds the target chewing cadence, the controller 418 may be configured to increase the vibration amplitude of the gastric vibrational therapy device 416. The increased vibration amplitude may provide enhanced mechanical stimulation to compensate for rapid eating behavior that may reduce natural satiety signaling. The magnitude of the amplitude increase may be proportional to the magnitude of the calculated deviation, enabling graduated therapeutic response to varying degrees of deviation from target eating behavior.

The operational parameters may also be modulated based on physiological data and risk assessment. When elevated baseline glucose or concerning glucose trends are detected before a meal, the controller 418 may increase vibration amplitude or extend stimulation duration to provide increased therapeutic intervention. When heart rate variability metrics indicate low vagal tone or sympathetic dominance, the controller 418 may adjust vibration frequency to frequencies associated with increased vagal activation. The modulation of operational parameters based on multiple input modalities may enable the system to provide adaptive therapeutic intervention that responds to both eating behavior and physiological state.

The operational parameters may also be modulated based on physiological data and risk assessment. When elevated baseline glucose or concerning glucose trends are detected before a meal, the controller 418 may increase vibration amplitude or extend stimulation duration to provide increased therapeutic intervention. When heart rate variability metrics indicate low vagal tone or sympathetic dominance, the controller 418 may adjust vibration frequency to frequencies associated with increased vagal activation. The modulation of operational parameters based on multiple input modalities may enable the system to provide adaptive therapeutic intervention that responds to both eating behavior and physiological state.

With reference to FIG. 4, the system may process acoustic signals from the chewing detection sensor 406 to identify chewing events using signal processing algorithms. When the chewing detection sensor 406 comprises an acoustic sensor configured to detect chewing sounds, the controller 418 may be configured to identify chewing events from the detected chewing sounds using signal processing algorithms including band-pass filtering and peak detection.

The acoustic sensor may capture audio signals containing chewing sounds generated during mastication. The captured audio signals may contain frequency components associated with chewing activity as well as background noise and other non-chewing sounds. The signal processing algorithms may isolate the frequency components associated with chewing activity from the captured audio signals to enable accurate identification of chewing events.

Band-pass filtering may be applied to the captured audio signals to isolate frequency components within a range associated with chewing sounds. Chewing produces characteristic sound patterns in a frequency range of approximately 500 Hz to 5000 Hz. The band-pass filter may attenuate frequency components below a lower cutoff frequency and frequency components above an upper cutoff frequency while passing frequency components within the band associated with chewing sounds. The band-pass filtering may remove low-frequency noise such as ambient room sounds and high-frequency noise such as electronic interference, improving the signal-to-noise ratio of the filtered audio signal. Band-pass filtering and peak detection are examples of signal processing algorithms that may be used to identify chewing events from detected chewing sounds. Other signal processing algorithms may also be employed, including but not limited to spectral analysis, envelope detection, wavelet transforms, and adaptive filtering techniques.

Spectral subtraction may be applied to the filtered audio signal to further reduce background noise. Spectral subtraction may estimate the noise spectrum from portions of the audio signal that do not contain chewing activity and subtract the estimated noise spectrum from the overall signal spectrum. The spectral subtraction may enhance the chewing-related components of the audio signal relative to residual background noise.

Feature extraction may be performed on the preprocessed audio signal to generate features suitable for chewing event detection. The feature extraction may include computation of a mel spectrogram, which represents the power spectrum of the audio signal on a mel frequency scale that approximates human auditory perception. The feature extraction may include computation of mel-frequency cepstral coefficients (MFCCs), which provide a compact representation of the spectral envelope of the audio signal. The mel spectrogram and MFCCs may capture spectral characteristics that distinguish chewing sounds from other sounds.

Peak detection may be applied to identify individual chewing events within the preprocessed audio signal. The peak detection may identify local maxima in the signal amplitude or in chewing probability values generated by a classifier. The peak detection may apply a threshold to distinguish chewing-related peaks from noise-related fluctuations. The peak detection may apply a minimum distance constraint between detected peaks to prevent multiple detections of a single chewing event. The minimum distance constraint may correspond to a minimum physiologically plausible interval between successive chews.

A classifier may be applied to sliding windows of the extracted features to generate chewing probability values for each window. The classifier may comprise a convolutional neural network or a recurrent neural network trained on labeled chewing audio data. The classifier may output a probability value indicating the likelihood that each window contains a chewing event. Windows with chewing probability values exceeding a threshold may be identified as containing chewing events.

The chewing cadence may be calculated from the identified chewing events using a sliding window approach. The sliding window approach may count the number of chewing events occurring within a time window of specified duration, such as 30 seconds. The chewing cadence may be calculated as the number of chewing events within the window divided by the window duration, converted to chews per minute. The sliding window may advance incrementally through the eating episode, providing updated chewing cadence values as new chewing events are detected.

The chewing cadence may be calculated using a sliding window approach with exponential smoothing to reduce noise. Exponential smoothing may be applied to the calculated chewing cadence values to reduce fluctuations caused by natural variation in chewing rhythm and detection noise. The exponential smoothing may compute a smoothed cadence value as a weighted combination of the current calculated cadence value and the previous smoothed cadence value. A smoothing factor may determine the relative weight given to the current calculated cadence value versus the previous smoothed cadence value. A lower smoothing factor may provide greater smoothing with slower response to changes in actual chewing cadence. A higher smoothing factor may provide less smoothing with faster response to changes in actual chewing cadence. The smoothed cadence value may provide a stable estimate of the user's current chewing cadence for comparison with the target chewing cadence and calculation of deviations.

The chewing detection sensor 406 may use sensor fusion combining multiple sensing modalities for robust detection of eating behavior. Sensor fusion refers to the combination of data from two or more sensors to achieve detection performance that exceeds the performance achievable with any single sensor alone. The sensor fusion approach may combine acoustic sensors and inertial sensors to detect chewing events through complementary sensing mechanisms. The acoustic sensors may detect chewing sounds generated during mastication while the inertial sensors may detect jaw movement patterns associated with chewing activity. The combination of acoustic and inertial sensing modalities may reduce false positive detections that may occur when relying on a single sensing modality.

The sensor fusion approach may employ temporal coincidence detection to identify chewing events that are detected by multiple sensing modalities within a specified time window. Temporal coincidence detection may require that a chewing event be detected by both the acoustic sensor and the inertial sensor within a time tolerance window to be classified as a confirmed chewing event. The time tolerance window may be approximately 100 milliseconds to account for slight timing differences between the acoustic signal generated by food mastication and the mechanical motion detected by the inertial sensor. Chewing events detected by only one sensing modality may be classified with lower confidence or may be excluded from the confirmed chewing event count depending on the detection confidence level of the single-modality detection.

The temporal coincidence detection may reduce false positive detections caused by non-chewing activities that may trigger one sensing modality but not the other. Talking may generate acoustic signals that resemble chewing sounds but may not produce the characteristic jaw movement patterns detected by the inertial sensor. Laughing may generate both acoustic signals and head movements but may not produce the rhythmic jaw closure patterns characteristic of chewing. The requirement for temporal coincidence across multiple sensing modalities may enable the system to distinguish chewing events from talking, laughing, and other non-chewing activities that may produce signals in one sensing modality.

The sensor fusion approach may calculate a fused chewing cadence from the confirmed chewing events detected through temporal coincidence. When sufficient confirmed chewing events are detected through temporal coincidence, the fused chewing cadence may be calculated from the inter-event intervals of the confirmed chewing events. When insufficient confirmed chewing events are detected through temporal coincidence, the system may fall back to the chewing cadence calculated from the sensing modality with higher detection confidence. The detection confidence for each sensing modality may be determined based on signal quality metrics, environmental noise levels, and historical detection accuracy for the current user.

The chewing detection sensor 406 may include swallow detection via throat-area acoustic monitoring. Swallowing produces characteristic acoustic signals that differ from chewing sounds in frequency content and temporal pattern. The characteristic swallow sound may include a brief acoustic burst associated with pharyngeal contraction followed by a quieter interval as the bolus passes through the esophagus. A microphone positioned in the throat area may detect the characteristic swallow sounds generated during deglutition. The throat-area acoustic monitoring may be implemented using a collar-mounted microphone, a neck-worn sensor patch, or an adhesive sensor positioned on the anterior neck surface.

The swallow detection via throat-area acoustic monitoring may employ signal processing algorithms to distinguish swallow sounds from other throat-area sounds including speech, coughing, and throat clearing. The signal processing algorithms may analyze the temporal envelope and spectral characteristics of detected acoustic events to classify events as swallows or non-swallows. Swallow sounds may exhibit a characteristic temporal pattern with a rapid onset, a brief duration of approximately 0.5 to 1.5 seconds, and a specific frequency content that differs from speech and coughing sounds. A classifier trained on labeled swallow audio data may generate probability values indicating the likelihood that each detected acoustic event corresponds to a swallow.

The chewing detection sensor 406 may include swallow detection via laryngeal motion detection using an accelerometer positioned on the throat. Swallowing involves elevation and anterior movement of the larynx as the hyoid bone and laryngeal cartilages move during the pharyngeal phase of deglutition. An accelerometer positioned on the anterior neck surface over the larynx may detect the characteristic motion pattern associated with laryngeal elevation during swallowing. The accelerometer may detect the acceleration associated with the upward and forward movement of the larynx at the initiation of the swallow and the subsequent return movement as the larynx descends following bolus passage.

The laryngeal motion detection may employ signal processing algorithms to identify swallow events from the accelerometer signal. The signal processing algorithms may detect peaks in the accelerometer signal corresponding to laryngeal elevation events. The signal processing algorithms may apply threshold detection and pattern matching to distinguish swallow-related laryngeal motion from other neck movements such as head turning, nodding, and speaking. The temporal pattern of laryngeal motion during swallowing may include a characteristic sequence of acceleration peaks that differs from the motion patterns associated with non-swallowing activities.

Swallow frequency may provide an additional eating pace metric that complements chewing cadence. The ratio of chews to swallows may indicate bite size, as larger bites may require more chews before swallowing while smaller bites may require fewer chews. The system may calculate a chews-per-swallow metric by dividing the number of detected chewing events by the number of detected swallow events over a specified time window. The chews-per-swallow metric may provide insight into eating behavior patterns that influence satiety signaling and metabolic responses. Users who swallow frequently with few chews per swallow may be consuming food rapidly with insufficient mastication, while users who chew extensively before each swallow may be eating at a pace that promotes satiety signaling.

With continued reference to FIG. 4, the continuous glucose monitor 408 may be configured to measure glucose levels of the user. The continuous glucose monitor 408 may be a wearable device with a subcutaneous sensor that measures interstitial glucose levels at regular intervals. The continuous glucose monitor 408 may measure glucose levels at intervals of 1-5 minutes, providing near-continuous monitoring of glucose dynamics throughout the day. The continuous glucose monitor 408 may provide glucose data including a current glucose level, a trend direction, and a rate of change of glucose levels.

The current glucose level may represent the most recent glucose measurement obtained from the continuous glucose monitor 408. The current glucose level may be expressed in milligrams per deciliter (mg/dL) or millimoles per liter (mmol/L). The current glucose level may indicate the user's glycemic state at the time of measurement and may be used to assess whether the user is in a normoglycemic, hyperglycemic, or hypoglycemic state.

The trend direction may indicate whether glucose levels are rising, falling, or stable over a recent time period. The trend direction may be determined by analyzing the trajectory of glucose measurements over a preceding time window. A rising trend may indicate that glucose levels are increasing, which may occur during or after food consumption as carbohydrates are absorbed and enter the bloodstream. A falling trend may indicate that glucose levels are decreasing, which may occur as insulin facilitates glucose uptake into cells. A stable trend may indicate that glucose levels are remaining relatively constant without substantial increase or decrease.

The rate of change may quantify the speed at which glucose levels are changing over time. The rate of change may be expressed in milligrams per deciliter per minute (mg/dL/min) or similar units. A positive rate of change may indicate rising glucose levels, with larger positive values indicating more rapid glucose elevation. A negative rate of change may indicate falling glucose levels, with larger negative values indicating more rapid glucose decline. The rate of change may provide information about the dynamics of glucose metabolism that complements the current glucose level measurement.

In the context of a method for adaptive control of gastric satiety therapy, acquiring physiological data may comprise receiving continuous glucose monitoring data including a glucose level and a glucose rate-of-change metric. The glucose level may correspond to the current glucose measurement obtained from the continuous glucose monitor 408. The glucose rate-of-change metric may correspond to the calculated rate at which glucose levels are changing over time. The continuous glucose monitoring data may be received wirelessly from the continuous glucose monitor 408 via a communication protocol such as Bluetooth.

The controller 418 may be configured to calculate a glucose-based adjustment factor based on the glucose levels received from the continuous glucose monitor 408. The glucose-based adjustment factor may be a numerical multiplier that modifies the operational parameters of the gastric vibrational therapy device 416 based on the user's current glycemic state. The glucose-based adjustment factor may increase the intensity of therapeutic intervention when glucose levels or glucose dynamics indicate elevated metabolic risk. The glucose-based adjustment factor may decrease the intensity of therapeutic intervention when glucose levels indicate stable or favorable glycemic conditions.

The controller 418 may calculate the glucose-based adjustment factor based on the current glucose level. When the current glucose level exceeds a first threshold, the controller 418 may calculate a glucose-based adjustment factor greater than 1.0 to increase therapeutic intervention intensity. The first threshold may be set at a glucose level indicating elevated baseline glucose, such as 110 mg/dL, 120 mg/dL, or 140 mg/dL. Higher current glucose levels may result in larger glucose-based adjustment factors. For example, a current glucose level exceeding 140 mg/dL may result in a glucose-based adjustment factor of 1.25, while a current glucose level between 120 mg/dL and 140 mg/dL may result in a glucose-based adjustment factor of 1.15, and a current glucose level between 110 mg/dL and 120 mg/dL may result in a glucose-based adjustment factor of 1.08.

The controller 418 may calculate the glucose-based adjustment factor based on the trend direction and rate of change. When the trend direction indicates rapidly rising glucose levels, the controller 418 may calculate a glucose-based adjustment factor that increases therapeutic intervention intensity. A rapidly rising trend may indicate that glucose is being absorbed quickly following food consumption, which may be associated with larger postprandial glucose excursions. The controller 418 may apply a multiplier to the glucose-based adjustment factor when the rate of change exceeds a threshold indicating rapid glucose elevation. For example, a rapidly rising trend may result in a multiplier of 1.20 applied to the glucose-based adjustment factor, while a moderately rising trend may result in a multiplier of 1.10.

The controller 418 may calculate the glucose-based adjustment factor based on glucose variability. Glucose variability may be calculated as the standard deviation of glucose measurements over a recent time period, such as the preceding hour. High glucose variability may indicate unstable glucose control and may be associated with increased metabolic risk. When glucose variability exceeds a threshold, the controller 418 may apply an additional multiplier to the glucose-based adjustment factor. For example, glucose variability exceeding 30 mg/dL may result in a multiplier of 1.12 applied to the glucose-based adjustment factor.

The controller 418 may apply the glucose-based adjustment factor to the operational parameters of the gastric vibrational therapy device 416. The operational parameters may include vibration frequency, vibration amplitude, duty cycle, and stimulation duration. The controller 418 may multiply one or more of the operational parameters by the glucose-based adjustment factor to generate adjusted operational parameters. The adjusted operational parameters may be transmitted to the gastric vibrational therapy device 416 to modulate the therapeutic intervention based on the user's glycemic state.

The controller 418 may apply the glucose-based adjustment factor to the vibration amplitude of the gastric vibrational therapy device 416. When the glucose-based adjustment factor is greater than 1.0, the controller 418 may increase the vibration amplitude proportionally to provide enhanced mechanical stimulation. The increased vibration amplitude may promote stronger activation of gastric mechanoreceptors and vagal afferent fibers, which may enhance satiety signaling and influence gastric emptying rate. When the glucose-based adjustment factor is less than 1.0, the controller 418 may decrease the vibration amplitude to provide reduced mechanical stimulation appropriate for favorable glycemic conditions.

The controller 418 may apply the glucose-based adjustment factor to the stimulation duration of the gastric vibrational therapy device 416. When the glucose-based adjustment factor indicates elevated metabolic risk, the controller 418 may extend the stimulation duration to provide prolonged therapeutic intervention. Extended stimulation duration may provide sustained activation of satiety signaling pathways throughout the postprandial period. The extended stimulation duration may be applied to post-meal therapy sessions to support glucose control during the period when postprandial glucose excursions occur.

The controller 418 may apply the glucose-based adjustment factor to the duty cycle of the gastric vibrational therapy device 416. When the glucose-based adjustment factor indicates elevated metabolic risk, the controller 418 may increase the duty cycle to provide a greater proportion of active stimulation time within each cycle period. An increased duty cycle may provide more continuous mechanical stimulation, which may enhance the cumulative effect of the therapeutic intervention on satiety signaling and gastric motility.

The controller 418 may cap the glucose-based adjustment factor at a maximum value to prevent excessive therapeutic intervention. The maximum value may be set at 1.40 or another value selected to balance therapeutic efficacy with user comfort and safety. The capping of the glucose-based adjustment factor may ensure that the adjusted operational parameters remain within acceptable ranges even when multiple glucose-related factors indicate elevated metabolic risk.

The controller 418 may monitor glucose response during an eating episode and adjust the glucose-based adjustment factor in real-time based on the observed glucose dynamics. The controller 418 may compare the current glucose level to a predicted glucose level based on the food properties and eating behavior detected during the eating episode. When the current glucose level exceeds the predicted glucose level by a threshold amount, the controller 418 may increase the glucose-based adjustment factor to provide compensatory therapeutic intervention. The real-time adjustment of the glucose-based adjustment factor may enable the system to respond to glucose dynamics that differ from predictions based on food properties and historical data.

The controller 418 may reduce or suspend therapeutic intervention when glucose levels indicate hypoglycemia risk. When the current glucose level falls below a hypoglycemia threshold, such as 70 mg/dL, the controller 418 may suspend operation of the gastric vibrational therapy device 416. The suspension of therapeutic intervention during hypoglycemia may prevent the gastric vibrational therapy device 416 from slowing gastric emptying when rapid glucose absorption is needed to restore normoglycemia. When the current glucose level is approaching the hypoglycemia threshold and the trend direction indicates falling glucose, the controller 418 may reduce the glucose-based adjustment factor to decrease therapeutic intervention intensity. The hypoglycemia protection functionality may be particularly relevant for users who take insulin or other glucose-lowering medications.

With continued reference to FIG. 4, the physiological monitoring device 407 may comprise the heart rate variability monitor 410 configured to measure heart rate variability metrics indicative of autonomic nervous system state. Heart rate variability (HRV) refers to the variation in time intervals between consecutive heartbeats. The heart rate variability monitor 410 may measure beat-to-beat heart rate intervals, also referred to as inter-beat intervals (IBI) or R-R intervals, which represent the time between successive R-waves in an electrocardiogram signal. The inter-beat intervals may be measured in milliseconds. The heart rate variability monitor 410 may acquire inter-beat interval data via a chest strap, a wrist-worn optical sensor, or a finger pulse oximeter.

The controller 418 may calculate heart rate variability metrics from the inter-beat intervals acquired by the heart rate variability monitor 410. The heart rate variability metrics may include time domain metrics and frequency domain metrics that characterize different aspects of heart rate variability and provide indicators of autonomic nervous system function. The time domain metrics may be calculated directly from the sequence of inter-beat intervals. The frequency domain metrics may be calculated from the power spectral density of the inter-beat interval time series.

The controller 418 may calculate RMSSD from the inter-beat intervals. RMSSD refers to the root mean square of successive differences between adjacent inter-beat intervals. RMSSD may be calculated by computing the differences between each pair of successive inter-beat intervals, squaring each difference, calculating the mean of the squared differences, and taking the square root of the mean. RMSSD may serve as a primary indicator of parasympathetic (vagal) activity. Higher RMSSD values may indicate greater parasympathetic influence on heart rate regulation, while lower RMSSD values may indicate reduced parasympathetic activity.

The controller 418 may calculate SDNN from the inter-beat intervals. SDNN refers to the standard deviation of normal-to-normal inter-beat intervals. SDNN may be calculated by computing the standard deviation of the inter-beat interval values over a measurement period. SDNN may reflect overall heart rate variability and may be influenced by both sympathetic and parasympathetic branches of the autonomic nervous system. SDNN may provide a general measure of autonomic function that captures both short-term and longer-term variations in heart rate.

The controller 418 may calculate pNN50 from the inter-beat intervals. pNN50 refers to the percentage of successive inter-beat interval differences that exceed 50 milliseconds. pNN50 may be calculated by counting the number of successive inter-beat interval differences greater than 50 milliseconds and dividing by the total number of successive differences, then multiplying by 100 to express the result as a percentage. pNN50 may serve as another indicator of parasympathetic activity, as the rapid beat-to-beat variations captured by pNN50 are primarily mediated by vagal influences on the sinoatrial node.

The controller 418 may calculate frequency domain metrics from the inter-beat intervals using power spectral density analysis. The power spectral density may be computed by resampling the inter-beat interval time series to a uniform sampling rate and applying spectral estimation techniques such as the Welch method. The power spectral density may represent the distribution of variance in the inter-beat interval signal across different frequency bands.

The controller 418 may calculate LF power from the power spectral density. LF power refers to the power in the low frequency band, which may span frequencies from approximately 0.04 Hz to 0.15 Hz. LF power may reflect both sympathetic and parasympathetic influences on heart rate variability, as well as baroreflex activity. The baroreflex is a homeostatic mechanism that regulates blood pressure through modulation of heart rate and vascular tone.

The controller 418 may calculate HF power from the power spectral density. HF power refers to the power in the high frequency band, which may span frequencies from approximately 0.15 Hz to 0.40 Hz. HF power may reflect parasympathetic (vagal) activity and respiratory sinus arrhythmia. Respiratory sinus arrhythmia refers to the natural variation in heart rate that occurs with breathing, wherein heart rate increases during inhalation and decreases during exhalation. The high frequency variations in heart rate associated with respiratory sinus arrhythmia are mediated primarily by vagal efferent activity.

The controller 418 may calculate LF/HF ratio from the LF power and HF power values. The LF/HF ratio may be calculated by dividing the LF power by the HF power. The LF/HF ratio may provide an indicator of sympatho-vagal balance. A higher LF/HF ratio may indicate greater sympathetic dominance relative to parasympathetic activity, which may be associated with stress states. A lower LF/HF ratio may indicate greater parasympathetic dominance, which may be associated with rest-and-digest states favorable for digestion and satiety signaling.

The controller 418 may calculate a vagal tone index from the inter-beat intervals. The vagal tone index may be a composite indicator of parasympathetic activity that combines multiple HRV metrics. The vagal tone index may be calculated by combining RMSSD and HF power in a normalized formula. The vagal tone index may provide a single metric that summarizes the overall level of vagal (parasympathetic) influence on heart rate regulation. Higher vagal tone index values may indicate stronger parasympathetic activity, while lower vagal tone index values may indicate reduced parasympathetic activity.

The controller 418 may determine an autonomic state based on HRV deviation from baseline. The baseline may be established through multiple resting HRV measurements collected over a period of one to two weeks. The baseline measurements may be collected during standardized conditions, such as morning measurements upon waking before arising from bed, to provide stable reference values. The baseline may include mean values and standard deviation values for RMSSD, HF power, LF/HF ratio, and vagal tone index.

The controller 418 may calculate deviation of current HRV metrics from the baseline values. The deviation may be expressed as a percentage change from the baseline mean value. For example, RMSSD deviation may be calculated as the difference between the current RMSSD value and the baseline RMSSD mean, divided by the baseline RMSSD mean, and multiplied by 100 to express the result as a percentage. Similar deviation calculations may be performed for vagal tone index and other HRV metrics.

The controller 418 may determine the autonomic state as low vagal tone when HRV metrics indicate suppressed parasympathetic activity. The low vagal tone state may be determined when RMSSD deviation is less than negative 30 percent or when vagal tone index deviation is less than negative 30 percent. The low vagal tone state may indicate that parasympathetic activity is substantially reduced compared to the user's baseline, which may be associated with stress, poor sleep, or other factors that suppress vagal function.

The controller 418 may determine the autonomic state as sympathetic dominant when HRV metrics indicate elevated sympathetic activity relative to parasympathetic activity. The sympathetic dominant state may be determined when the LF/HF ratio exceeds a threshold value, such as 2.5. The sympathetic dominant state may indicate a stress state wherein sympathetic nervous system activity predominates over parasympathetic activity. The sympathetic dominant state may be associated with fight-or-flight responses and may not be favorable for digestion and satiety signaling.

The controller 418 may determine the autonomic state as high vagal tone when HRV metrics indicate elevated parasympathetic activity. The high vagal tone state may be determined when RMSSD deviation exceeds positive 20 percent. The high vagal tone state may indicate that parasympathetic activity is elevated compared to the user's baseline, which may represent favorable conditions for digestion and satiety signaling. The high vagal tone state may be associated with relaxed states and may indicate that the autonomic nervous system is in a rest-and-digest mode.

The controller 418 may determine the autonomic state as balanced when HRV metrics do not indicate substantial deviation from baseline in either the parasympathetic or sympathetic direction. The balanced state may be determined when RMSSD deviation is within a range that does not meet the criteria for low vagal tone or high vagal tone, and when the LF/HF ratio does not exceed the threshold for sympathetic dominant classification. The balanced state may indicate that the autonomic nervous system is functioning within normal parameters for the user.

The determined autonomic state may inform therapeutic protocol adjustments. When the autonomic state is determined to be low vagal tone or sympathetic dominant, the controller 418 may adjust operational parameters of the gastric vibrational therapy device 416 to provide enhanced therapeutic intervention. When the autonomic state is determined to be high vagal tone, the controller 418 may adjust operational parameters to provide reduced therapeutic intervention, as the favorable autonomic conditions may support natural satiety signaling without enhanced intervention. When the autonomic state is determined to be balanced, the controller 418 may apply standard therapeutic protocols without substantial adjustment based on autonomic state.

With continued reference to FIG. 4, the system may further comprise an image capture device 402 configured to acquire images of food and an image analysis module 404 configured to determine food properties from the acquired images. The controller 418 may be further configured to determine the target chewing cadence based on the food properties determined by the image analysis module 404. The integration of food image analysis with chewing cadence determination may enable the system to adjust target eating parameters based on characteristics of the food being consumed.

The image capture device 402 may acquire images of food before consumption by the user. The image capture device 402 may be configured to capture images from an overhead angle to facilitate accurate portion size estimation. The image capture device 402 may provide guidance to the user regarding image capture, including prompts for reference object placement, lighting optimization, and multiple angle capture for complex food presentations. The acquired images may be transmitted to the image analysis module 404 for processing and food property extraction.

The image capture device 402 may comprise a wearable camera integrated into smart glasses. The smart glasses may include a forward-facing camera positioned to capture images of food placed in front of the user. The wearable camera integrated into smart glasses may enable hands-free image capture, allowing the user to photograph food without manipulating a separate device. The smart glasses configuration may provide a convenient form factor for capturing food images at the beginning of eating episodes.

The image capture device 402 may comprise a camera integrated into dining area infrastructure for restaurant-based systems. The camera integrated into dining area infrastructure may be positioned to capture images of food served to patrons at dining tables. The restaurant-based system may enable automated food image capture without requiring user action, as the infrastructure camera may detect food placement and capture images automatically. The restaurant-based implementation may be suitable for commercial food service environments where consistent food image capture is desired across multiple patrons.

The image analysis module 404 may use convolutional neural networks trained on food image databases to identify foods present in acquired images. Convolutional neural networks are a class of deep learning models that apply convolutional filters to image data to extract hierarchical features for classification and detection tasks. The convolutional neural networks may be trained on food image databases containing images of food items with associated labels indicating food identity. The food image databases may include databases such as Food-101, UNIMIB2016, and proprietary databases containing images across thousands of food categories.

The image analysis module 404 may generate confidence scores for identified foods. The confidence scores may represent the probability that the identified food label correctly corresponds to the food item depicted in the image. Higher confidence scores may indicate greater certainty in the food identification, while lower confidence scores may indicate ambiguity or uncertainty in the identification. The confidence scores may be used to determine whether user confirmation is needed for ambiguous identifications.

The image analysis module 404 may generate bounding boxes localizing each food item within the acquired image. The bounding boxes may define rectangular regions within the image that contain individual food items. The bounding boxes may enable the image analysis module 404 to separately analyze multiple food items present in a single image. The bounding boxes may also facilitate portion size estimation by defining the spatial extent of each food item within the image.

The image analysis module 404 may provide hierarchical classification of identified foods. The hierarchical classification may include general food categories, subcategories, and specific food items. For example, a hierarchical classification may progress from a general category such as pasta to a subcategory such as white pasta to a specific food item such as fettuccine alfredo. The hierarchical classification may enable the system to apply food property estimates at an appropriate level of specificity based on the confidence of the identification.

The food properties determined by the image analysis module 404 may comprise at least one of food texture classification, estimated caloric content, macronutrient composition, or glycemic index. The food properties may be determined from the identified foods using nutritional databases and texture analysis algorithms. The determined food properties may inform the target chewing cadence and therapeutic protocol parameters for the eating episode.

The image analysis module 404 may classify food texture into categories including very soft, soft, moderate, firm, and very firm based on texture features. The texture classification may be performed using convolutional neural networks trained on texture features extracted from food images. The texture features may include edge patterns, surface characteristics, and visual indicators of food consistency. The five-level texture classification may enable the system to categorize foods across a spectrum of chewing requirements.

The image analysis module 404 may classify food texture into categories including very soft, soft, moderate, firm, and very firm based on texture features. The texture classification may be performed using convolutional neural networks trained on texture features extracted from food images. The texture features may include edge patterns, surface characteristics, and visual indicators of food consistency. The five-level texture classification may enable the system to categorize foods across a spectrum of chewing requirements.

The very soft texture category may include foods that require minimal chewing, such as smoothies, milkshakes, protein shakes, yogurt, pudding, ice cream, mashed potatoes, pureed soups, and soft breads without crust. Foods in the very soft texture category may be associated with very low mechanical satiety stimulation and high overconsumption risk due to the minimal chewing effort required for consumption.

The soft texture category may include foods that require low chewing effort, such as pasta, white rice, oatmeal, ripe bananas, cooked vegetables, soft cheeses, and scrambled eggs. Foods in the soft texture category may be associated with low mechanical satiety stimulation and a tendency toward faster eating compared to firmer foods.

The moderate texture category may include foods that require standard chewing effort, such as cooked chicken breast, fish, most cooked vegetables, most fruits including apples and oranges, and whole grain breads. Foods in the moderate texture category may be associated with adequate mechanical satiety stimulation when consumed at a normal eating pace.

The firm texture category may include foods that require substantial chewing effort, such as steak, pork chops, raw vegetables including carrots and celery, nuts, seeds, crusty breads, and bagels. Foods in the firm texture category may be associated with high mechanical satiety stimulation and a naturally slower eating pace due to the chewing effort required.

The very firm texture category may include foods that require extensive chewing effort, such as tough cuts of meat, hard raw vegetables, whole nuts, and very chewy foods. Foods in the very firm texture category may be associated with maximum mechanical satiety stimulation and a very slow natural eating pace.

The moderate texture category may include foods that require standard chewing effort, such as cooked chicken breast, fish, most cooked vegetables, most fruits including apples and oranges, and whole grain breads. Foods in the moderate texture category may be associated with adequate mechanical satiety stimulation when consumed at a normal eating pace.

The firm texture category may include foods that require substantial chewing effort, such as steak, pork chops, raw vegetables including carrots and celery, nuts, seeds, crusty breads, and bagels. Foods in the firm texture category may be associated with high mechanical satiety stimulation and a naturally slower eating pace due to the chewing effort required.

The very firm texture category may include foods that require extensive chewing effort, such as tough cuts of meat, hard raw vegetables, whole nuts, and very chewy foods. Foods in the very firm texture category may be associated with maximum mechanical satiety stimulation and a very slow natural eating pace.

The image analysis module 404 may estimate portion size using reference object scaling by detecting reference objects of known size in the image. Reference objects may include utensils, plates, credit cards, or the user's hand. The image analysis module 404 may detect the reference object within the acquired image and calculate a pixels-to-centimeters conversion factor based on the known dimensions of the reference object. The image analysis module 404 may estimate the area and depth of food items using the conversion factor to calculate food volume. The calculated food volume may be converted to food weight using food-specific density values.

The image analysis module 404 may estimate portion size using depth estimation from dual-camera systems or structured light for three-dimensional (3D) reconstruction. Dual-camera systems may include stereoscopic camera configurations that capture images from two slightly different viewpoints to enable depth calculation through triangulation. Structured light systems may project known light patterns onto the food surface and analyze the deformation of the projected patterns to calculate depth information. The depth estimation may enable the image analysis module 404 to generate a depth map representing the three-dimensional surface of the food. The depth map may be used for 3D reconstruction of food volume, which may provide more accurate portion size estimation compared to two-dimensional image analysis methods.

The image analysis module 404 may query nutritional databases using the identified foods and estimated portions to determine nutritional properties. The nutritional databases may include databases such as the United States Department of Agriculture (USDA) FoodData Central and proprietary nutritional databases. The nutritional properties may include total calories, macronutrient composition including protein, carbohydrates, fat, and fiber content, glycemic index, glycemic load, and caloric density.

The estimated caloric content may represent the total caloric value of the identified foods based on the estimated portion sizes. The estimated caloric content may be calculated by summing the caloric contributions of each identified food item, with each contribution determined by multiplying the caloric density of the food by the estimated portion weight.

The macronutrient composition may include protein content, carbohydrate content, fat content, and fiber content expressed in grams and as percentages of total calories. The macronutrient composition may inform the expected metabolic response to the meal and may influence the target chewing cadence and therapeutic protocol parameters.

The glycemic index may represent the expected blood glucose response to the carbohydrate content of the identified foods. The glycemic index may be expressed as a value on a scale where low glycemic index foods have values below 55, medium glycemic index foods have values between 56 and 69, and high glycemic index foods have values above 70. The glycemic index may be calculated as a weighted average based on the carbohydrate contribution of each identified food item. Foods with high glycemic index values may be associated with rapid glucose elevation following consumption.

The glycemic load may represent the overall glycemic impact of the meal accounting for both the glycemic index and the quantity of carbohydrates consumed. The glycemic load may be calculated by multiplying the glycemic index by the carbohydrate content in grams and dividing by 100. The glycemic load may provide a more comprehensive indicator of expected glucose response than glycemic index alone, as the glycemic load accounts for portion size in addition to carbohydrate quality.

The controller 418 may determine the target chewing cadence based on the food texture classification. The target chewing cadence may be inversely proportional to food texture firmness. Soft foods may be assigned a lower target chewing cadence to compensate for reduced mechanical satiety stimulation, while firm foods may be assigned a higher target chewing cadence as the texture naturally slows eating pace. The texture-responsive target chewing cadence determination may enable the system to adjust eating pace targets based on the satiety-promoting characteristics of the food being consumed.

With continued reference to FIG. 4, the machine learning engine 420 may comprise multiple specialized computational models configured to learn from historical meal data and generate predictions for glucose responses, eating behaviors, and therapeutic protocol optimization. The machine learning engine 420 may implement a multi-stream neural network architecture with separate processing streams for different input modalities. The multi-stream neural network architecture may include a first stream configured to process chewing data, a second stream configured to process physiologic data, and a third stream configured to process image data. Each stream may comprise neural network layers configured to extract features from the corresponding input modality.

The first stream of the multi-stream neural network may receive chewing data from a chewing detection sensor 406. The chewing data may include time-series data representing chewing events, chewing cadence values, inter-chew intervals, and swallow timing information. The first stream may comprise recurrent neural network layers or convolutional neural network layers configured to process the temporal patterns in the chewing data time-series. The first stream may extract features representing chewing rhythm, chewing consistency, eating pace variations, and other characteristics of eating behavior captured in the chewing data.

The second stream of the multi-stream neural network may receive physiologic data from physiological monitoring devices. The physiologic data may include glucose levels, glucose rate-of-change values, heart rate variability metrics, sleep quality scores, activity level data, and stress indicators. The second stream may comprise fully connected neural network layers or recurrent neural network layers configured to process the physiologic data. The second stream may extract features representing metabolic state, autonomic nervous system balance, recovery status, and other physiological characteristics relevant to satiety and glucose regulation.

The third stream of the multi-stream neural network may receive image data from an image capture device 402 and image analysis module 404. The image data may include food property features extracted from food images, such as texture classification values, estimated caloric content, macronutrient composition values, and glycemic index estimates. The third stream may comprise fully connected neural network layers configured to process the food property features. The third stream may extract features representing meal characteristics that influence expected metabolic responses and appropriate eating pace targets.

The outputs of the first stream, the second stream, and the third stream may be fused using an attention-based mechanism. The attention-based mechanism may compute attention weights that determine the relative contribution of each stream to the fused output. The attention weights may be learned during training and may vary based on the input data, enabling the multi-stream neural network to dynamically adjust the importance assigned to each input modality based on the current context. The attention-based mechanism may enable the multi-stream neural network to focus on the most informative input modalities for a given prediction task while still incorporating information from all available modalities.

The attention-based mechanism may compute attention scores for each stream based on the extracted features. The attention scores may be computed using a learned attention function that maps the extracted features to scalar attention values. The attention scores may be normalized using a softmax function to produce attention weights that sum to one across the streams. The normalized attention weights may be applied to the stream outputs to produce weighted stream outputs. The weighted stream outputs may be combined through concatenation or summation to produce a fused representation that incorporates information from all input modalities with learned weighting.

The fused representation produced by the attention-based mechanism may be processed by additional neural network layers to generate predictions. The additional neural network layers may comprise fully connected layers configured to map the fused representation to output values. The output values may include a predicted satiety-failure score, a predicted glucose response, a predicted chewing cadence, and recommended therapeutic protocol parameters. The predicted satiety-failure score may represent a probability metric indicating likelihood of overconsumption during an eating episode based on the fused analysis of chewing behavior, physiologic state, and food properties.

The machine learning engine 420 may implement gradient boosting models for glucose prediction. Gradient boosting is an ensemble learning technique that combines multiple weak prediction models, typically decision trees, into a strong prediction model through iterative optimization. The gradient boosting models may be trained on historical meal data to predict glucose responses based on input features including food properties, physiological state parameters, eating behavior metrics, and contextual factors.

The gradient boosting models may implement feature importance analysis to identify which input features contribute most strongly to glucose prediction accuracy. Feature importance analysis may quantify the contribution of each input feature to the prediction performance of the gradient boosting model. The feature importance values may be calculated based on the frequency with which each feature is used for splitting decisions in the decision trees, the improvement in prediction accuracy achieved by splits on each feature, or the decrease in prediction accuracy when each feature is permuted or removed.

The feature importance analysis may enable the machine learning engine 420 to discover individual sensitivity profiles for each user. The individual sensitivity profiles may indicate which input features have the greatest influence on glucose responses for a specific user. Some users may exhibit high sensitivity to sleep quality, wherein sleep deprivation produces large increases in glucose excursions. Other users may exhibit high sensitivity to stress levels, wherein elevated stress produces substantial glucose impairment. Other users may exhibit high sensitivity to menstrual cycle phase, wherein certain cycle phases are associated with altered glucose regulation. The feature importance analysis may identify these individual sensitivity patterns from the historical meal data for each user.

The machine learning engine 420 may implement transfer learning to fine-tune parameters for a specific user. Transfer learning is a machine learning technique wherein a model trained on a first dataset is adapted for use on a second dataset by fine-tuning model parameters using data from the second dataset. The machine learning engine 420 may use transfer learning to adapt a population-level model trained on data from multiple users to the specific characteristics of an individual user.

The population-level model may be trained on historical meal data aggregated from a population of users. The population-level model may learn general relationships between input features and glucose responses that apply across the user population. The population-level model may provide a starting point for personalized prediction that captures common patterns in glucose regulation and eating behavior.

The transfer learning process may fine-tune the parameters of the population-level model using historical meal data from the specific user. The fine-tuning may adjust the model parameters to better capture the individual characteristics of the specific user while retaining the general knowledge learned from the population data. The fine-tuning may be performed using a smaller learning rate than the initial training to preserve the general knowledge while adapting to user-specific patterns.

The transfer learning approach may enable the machine learning engine 420 to provide personalized predictions with limited user-specific data. New users may initially receive predictions based primarily on the population-level model. As the machine learning engine 420 collects historical meal data from the new user, the transfer learning process may progressively fine-tune the model parameters to improve prediction accuracy for the specific user. The transfer learning approach may enable the machine learning engine 420 to achieve personalized prediction performance more rapidly than training a user-specific model from scratch.

The machine learning engine 420 may use transfer learning to adapt a population-level model to fine-tune parameters for a specific user. Transfer learning is a machine learning technique wherein a model trained on a first dataset is adapted for use on a second dataset by fine-tuning model parameters using data from the second dataset. The machine learning engine 420 may use transfer learning to adapt a population-level model trained on data from multiple users to the specific characteristics of an individual user.

The population-level model may be trained on historical meal data aggregated from a population of users. The population-level model may learn general relationships between input features and glucose responses that apply across the user population. The population-level model may provide a starting point for personalized prediction that captures common patterns in glucose regulation and eating behavior.

The transfer learning process may fine-tune the parameters of the population-level model using historical meal data from the specific user. The fine-tuning may adjust the model parameters to better capture the individual characteristics of the specific user while retaining the general knowledge learned from the population data. The fine-tuning may be performed using a smaller learning rate than the initial training to preserve the general knowledge while adapting to user-specific patterns.

The transfer learning approach may enable the machine learning engine 420 to provide personalized predictions with limited user-specific data. New users may initially receive predictions based primarily on the population-level model. As the machine learning engine 420 collects historical meal data from the new user, the transfer learning process may progressively fine-tune the model parameters to improve prediction accuracy for the specific user. The transfer learning approach may enable the machine learning engine 420 to achieve personalized prediction performance more rapidly than training a user-specific model from scratch.

The machine learning engine 420 may identify synergistic interaction effects between input features through analysis of historical meal data. Synergistic interaction effects may occur when combinations of input features produce effects that exceed the sum of the individual feature effects. The machine learning engine 420 may discover interaction effects such as sleep deprivation combined with elevated stress producing glucose impairment greater than the additive prediction from sleep deprivation alone and stress alone. The discovered interaction effects may be incorporated into the prediction models to improve accuracy when multiple risk factors are present simultaneously.

The machine learning engine 420 may continuously learn from new meal data as the user continues to use the system. Each completed eating episode may provide new training data including the input features, the predictions made, the therapeutic protocol delivered, and the actual outcomes observed. The machine learning engine 420 may periodically retrain or update the prediction models using the accumulated historical meal data. The continuous learning may enable the machine learning engine 420 to adapt to changes in user characteristics over time and to improve prediction accuracy as more user-specific data becomes available.

The machine learning engine 420 may discover individual sensitivity profiles for each user through analysis of historical meal data and feature importance quantification. The individual sensitivity profiles may characterize which input factors have the greatest influence on metabolic outcomes for a specific user. The machine learning engine 420 may classify users into phenotype categories based on the discovered sensitivity profiles, including sleep-sensitive phenotype, stress-reactive phenotype, cycle-driven phenotype, and balanced phenotype.

The sleep-sensitive phenotype may be assigned to users for whom sleep quality and sleep duration have disproportionately large effects on glucose responses and eating behavior outcomes. Users with the sleep-sensitive phenotype may exhibit substantially larger glucose excursions following meals consumed after nights of poor sleep compared to meals consumed after nights of adequate sleep. The machine learning engine 420 may identify the sleep-sensitive phenotype when feature importance analysis indicates that sleep-related input features rank among the highest contributors to prediction accuracy for a specific user, with sleep feature importance values exceeding a threshold relative to other input features.

The stress-reactive phenotype may be assigned to users for whom stress levels and autonomic state indicators have disproportionately large effects on metabolic outcomes. Users with the stress-reactive phenotype may exhibit substantially impaired glucose regulation during periods of elevated stress compared to periods of low stress. The machine learning engine 420 may identify the stress-reactive phenotype when feature importance analysis indicates that stress-related input features, including heart rate variability metrics and galvanic skin response measurements, rank among the highest contributors to prediction accuracy for a specific user.

The cycle-driven phenotype may be assigned to female users for whom menstrual cycle phase has disproportionately large effects on glucose responses and appetite regulation. Users with the cycle-driven phenotype may exhibit substantially different metabolic responses during different phases of the menstrual cycle, with certain phases associated with elevated glucose excursions or altered satiety signaling. The machine learning engine 420 may identify the cycle-driven phenotype when feature importance analysis indicates that menstrual cycle phase input features rank among the highest contributors to prediction accuracy for a specific user.

The balanced phenotype may be assigned to users for whom no single input factor category dominates the prediction accuracy. Users with the balanced phenotype may exhibit relatively uniform sensitivity across multiple input factor categories, with sleep quality, stress levels, activity patterns, and other factors contributing comparably to metabolic outcomes. The machine learning engine 420 may identify the balanced phenotype when feature importance analysis indicates that no single input feature category substantially exceeds other categories in contribution to prediction accuracy.

The phenotype classification may inform therapeutic protocol personalization. Users classified with the sleep-sensitive phenotype may receive increased therapeutic intervention following nights of poor sleep, with the system applying larger adjustments to operational parameters based on sleep quality data. Users classified with the stress-reactive phenotype may receive increased therapeutic intervention during periods of elevated stress, with the system applying larger adjustments based on heart rate variability metrics and stress indicators. Users classified with the cycle-driven phenotype may receive cycle-phase-specific therapeutic protocols, with the system adjusting intervention intensity based on current menstrual cycle phase. Users classified with the balanced phenotype may receive therapeutic protocols that apply moderate adjustments across multiple input factor categories without emphasizing any single factor.

The machine learning engine 420 may identify synergistic interaction effects where combinations of input factors produce effects on metabolic outcomes that exceed simple additive prediction from the individual factors. Additive prediction assumes that the combined effect of multiple factors equals the sum of the individual factor effects. Synergistic interaction effects occur when the combined effect exceeds the additive prediction, indicating that the factors interact in a non-linear manner that amplifies the overall effect.

The machine learning engine 420 may discover synergistic interaction effects through analysis of prediction residuals in historical meal data. Prediction residuals represent the difference between predicted outcomes and actual observed outcomes. When the machine learning engine 420 observes that prediction residuals are systematically larger when multiple input factors are present simultaneously compared to when factors are present individually, the machine learning engine 420 may identify a synergistic interaction effect between those factors. The discovery of synergistic interaction effects may enable the machine learning engine 420 to improve prediction accuracy by incorporating interaction terms that capture non-additive effects of factor combinations. The controller 418 may auto-trigger the gastric vibrational therapy device 416 a specified amount of time after the chewing detection sensor 406 detects eating onset. The specified amount of time may be a configurable parameter that determines the delay between eating onset detection and automatic activation of the gastric vibrational therapy device 416. The specified amount of time may be set to a default value, such as 2 minutes, 5 minutes, or 10 minutes after eating onset. The auto-trigger functionality may enable the system to initiate gastric vibrational therapy automatically without requiring manual user input to activate the gastric vibrational therapy device 416 at the beginning of each eating episode. The automatic activation may improve therapy compliance by reducing the burden on users to remember to activate the gastric vibrational therapy device 416 manually. Some users may prefer earlier therapy activation to provide pre-meal priming before substantial food consumption occurs during the eating episode. Other users may prefer later therapy activation to allow time for natural eating rhythm establishment before intervention. The controller 418 may provide user-configurable settings for the specified time period, enabling personalization of the auto-trigger timing.

The specified time period may be adjusted automatically based on detected eating behavior patterns and therapeutic outcomes. The controller 418 may analyze historical data to determine whether earlier or later therapy activation is associated with better outcomes for a specific user. If earlier therapy activation is associated with improved compliance with target chewing cadence or reduced glucose excursions, the controller 418 may automatically reduce the specified time period. If later therapy activation is associated with better outcomes, the controller 418 may automatically increase the specified time period. The automatic adjustment may optimize the auto-trigger timing based on observed effectiveness for each user.

The user interface module 424 may provide a pre-meal briefing displaying a risk score, a protocol, predictions, and recommendations to the user before an eating episode begins. The pre-meal briefing may be presented on a display screen of a mobile device or other electronic device hosting the user interface module 424. The pre-meal briefing may provide the user with information regarding the assessed metabolic risk for the upcoming eating episode and the therapeutic intervention that will be applied based on the assessed risk.

The pre-meal briefing may display the risk score calculated by the controller 418 based on physiological data, food properties, temporal context, and behavioral context. The risk score may be displayed as a numerical value, such as a value ranging from 0 to 100, or as a categorical indicator such as low risk, moderate risk, or high risk. The display of the risk score may enable the user to understand the overall metabolic risk assessment for the upcoming eating episode. The pre-meal briefing may include a visual representation of the risk score, such as a color-coded indicator where green represents low risk, yellow represents moderate risk, and red represents high risk.

The controller 418 may calculate the risk score as a weighted combination of multiple physiological parameters obtained from the physiological monitoring device 407. The risk score calculation may incorporate factors including current glucose level, glucose rate-of-change, glucose variability, heart rate variability metrics such as RMSSD and LF/HF ratio, sleep quality score from the preceding night, recent activity level, stress indicators from galvanic skin response measurements, and menstrual cycle phase for applicable users. Each factor may be assigned a weight reflecting its relative contribution to metabolic risk. For example, a glucose level exceeding 140 mg/dL may contribute 15 points to the risk score, a rapidly rising glucose trend may contribute 10 points, an RMSSD value below 25 milliseconds indicating low vagal tone may contribute 12 points, and a sleep quality score below 60% may contribute 8 points. The individual factor contributions may be summed to produce an aggregate risk score ranging from 0 to 100, where higher values indicate greater metabolic risk. Alternatively, the risk score may be categorized into discrete levels such as low risk corresponding to scores of 0-30, moderate risk corresponding to scores of 31-60, and high risk corresponding to scores of 61-100. The machine learning engine 420 may refine the risk score calculation over time by analyzing historical meal data to identify which factors are most predictive of adverse metabolic outcomes for each individual user.

The controller 418 may calculate a satiety-failure score representing a probability metric indicating likelihood of overconsumption during an eating episode. The satiety-failure score may integrate multiple factors including chewing rate deviation from the target chewing cadence, glucose rate-of-change metric, autonomic tone metric derived from heart rate variability, and meal duration. The satiety-failure score may be calculated as a weighted combination of these factors, with weights determined through machine learning analysis of historical meal data. A higher satiety-failure score may indicate greater likelihood of overconsumption, while a lower satiety-failure score may indicate that the user is eating in a manner consistent with satiety signaling. The controller 418 may modulate the operational parameters of the gastric vibrational therapy device 416 based on the satiety-failure score, increasing therapy intensity when the satiety-failure score exceeds a first threshold and decreasing therapy intensity when the satiety-failure score falls below a second threshold.

The pre-meal briefing may display the therapeutic protocol that will be applied during the eating episode. The protocol display may include information regarding pre-meal priming duration, target chewing cadence, gastric vibrational therapy parameters including vibration frequency and vibration amplitude, and planned post-meal therapy duration. The protocol display may enable the user to understand the therapeutic intervention that has been determined based on the comprehensive risk assessment. The protocol display may include explanatory text describing the rationale for the selected protocol parameters.

The pre-meal briefing may display predictions generated by a machine learning engine 420 regarding expected outcomes for the eating episode. The predictions may include a predicted peak glucose value, a predicted glucose excursion, a predicted eating duration, and a predicted compliance likelihood. The display of predictions may enable the user to understand the expected metabolic response to the upcoming meal based on the food properties and current physiological state. The predictions may be displayed with confidence intervals or uncertainty ranges to indicate the precision of the predictions.

The pre-meal briefing may display recommendations for the user regarding eating behavior and food choices. The recommendations may include suggestions for eating pace, such as a recommendation to eat slowly when elevated risk is detected. The recommendations may include suggestions for food modifications, such as a recommendation to reduce portion size or to add fiber-rich foods to the meal when high glycemic index foods are detected. The recommendations may include suggestions for pre-meal activities, such as a recommendation to take a brief walk before eating when prolonged sedentary time is detected. The recommendations may be personalized based on the user's historical responses and discovered sensitivity profiles.

The user interface module 424 may provide a real-time eating progress display showing eating duration, current pace, target pace, and deviation percentage during an eating episode. The real-time eating progress display may be presented on a display screen of the electronic device 304 that the user may view during eating. The real-time eating progress display may provide continuous feedback regarding eating behavior as the eating episode progresses.

The real-time eating progress display may show eating duration as the elapsed time since the eating episode began. The eating duration may be displayed as minutes and seconds elapsed since eating onset was detected or since the user indicated meal start. The eating duration display may enable the user to monitor how long the eating episode has been in progress and to compare the current duration to typical meal durations.

The real-time eating progress display may show current pace as the chewing cadence detected by a chewing detection sensor 406. The current pace may be displayed as chews per minute or as a qualitative indicator such as slow, moderate, or fast. The current pace display may be updated at regular intervals, such as every 5 seconds or every 10 seconds, to provide near-real-time feedback on eating behavior. The current pace display may enable the user to monitor actual eating speed during the eating episode.

The real-time eating progress display may show target pace as the target chewing cadence determined by the controller 418 based on physiological data and food properties. The target pace may be displayed as chews per minute or as a qualitative indicator. The target pace display may enable the user to understand the eating pace that has been determined as appropriate for the current eating episode based on the comprehensive assessment of physiological state and food characteristics.

The real-time eating progress display may show deviation percentage as the calculated deviation between the current pace and the target pace expressed as a percentage. The deviation percentage may be calculated by dividing the difference between the current pace and the target pace by the target pace and multiplying by 100. A positive deviation percentage may indicate that the user is eating faster than the target pace, while a negative deviation percentage may indicate that the user is eating slower than the target pace. The deviation percentage display may enable the user to understand the magnitude and direction of departure from the target eating pace.

The real-time eating progress display may include visual indicators that communicate eating pace status at a glance. The visual indicators may include color-coded elements where green indicates eating pace within an acceptable range of the target pace, yellow indicates moderate deviation from the target pace, and red indicates substantial deviation from the target pace. The visual indicators may include graphical elements such as a speedometer-style gauge, a progress bar, or an animated icon that reflects current eating pace relative to the target pace. The visual indicators may enable the user to quickly assess eating pace status without reading numerical values.

The user interface module 424 may prompt the user for subjective ratings including a hunger rating before a meal and fullness and satisfaction ratings after a meal. The subjective rating prompts may collect self-reported data regarding the user's internal states that complement the objective sensor data collected by the system. The subjective ratings may be used by a machine learning engine 420 to improve prediction accuracy and to personalize therapeutic protocols based on the relationship between subjective states and metabolic outcomes.

The user interface module 424 may prompt the user for a hunger rating before the meal begins. The hunger rating prompt may be presented when the user initiates a meal logging session or when eating onset is detected. The hunger rating may be collected using a numerical scale, such as a scale from 1 to 10 where 1 represents not hungry at all and 10 represents extremely hungry. The hunger rating may be collected using a visual analog scale where the user positions a slider along a continuum from not hungry to extremely hungry. The hunger rating may be collected using categorical options such as not hungry, slightly hungry, moderately hungry, very hungry, and extremely hungry.

The pre-meal hunger rating may inform therapeutic protocol adjustments. When the user reports high hunger, the controller 418 may increase therapeutic intervention intensity to provide enhanced satiety support during the eating episode. When the user reports low hunger, the controller 418 may apply standard therapeutic protocols or may prompt the user to consider whether eating is driven by hunger or by other factors such as habit, social pressure, or emotional state. The pre-meal hunger rating may be stored in memory 422 and associated with the meal record for use in machine learning model training.

The user interface module 424 may prompt the user for a fullness rating after the meal is completed. The fullness rating prompt may be presented when the user indicates meal completion or when eating cessation is detected. The fullness rating may be collected using a numerical scale, such as a scale from 1 to 10 where 1 represents not full at all and 10 represents extremely full or uncomfortably full. The fullness rating may be collected using a visual analog scale or categorical options similar to the hunger rating. The fullness rating may capture the user's subjective perception of satiety following the eating episode.

The user interface module 424 may prompt the user for a satisfaction rating after the meal is completed. The satisfaction rating may capture the user's subjective assessment of meal enjoyment and eating experience quality. The satisfaction rating may be collected using a numerical scale, a visual analog scale, or categorical options. The satisfaction rating may be collected separately from the fullness rating to distinguish between physical satiety and psychological satisfaction with the eating experience. The satisfaction rating may inform personalization of therapeutic protocols to balance metabolic objectives with eating experience quality.

The post-meal fullness and satisfaction ratings may be stored in memory 422 and associated with the meal record. The stored ratings may be used by a machine learning engine 420 to analyze relationships between eating behavior, therapeutic intervention, and subjective outcomes. The machine learning engine 420 may identify patterns indicating which therapeutic protocols and eating behaviors are associated with higher fullness ratings and higher satisfaction ratings. The discovered patterns may inform protocol optimization to achieve both metabolic objectives and positive subjective eating experiences.

The user interface module 424 may generate and display meal reports with glucose response analysis and personalized recommendations delivered hours after meal completion. The delayed meal report generation may enable the system to incorporate postprandial glucose response data that becomes available in the hours following meal consumption. The delayed delivery may provide the user with meal analysis after sufficient time has elapsed to observe the complete glucose response curve.

The meal report may be generated at a specified time following meal completion, such as 2 hours, 3 hours, or 4 hours after the eating episode ended. The specified time may be selected to allow observation of the postprandial glucose response including the peak glucose value, the return toward baseline, and the 2-hour glucose value. The meal report may be delivered as a notification on the user's mobile device that prompts the user to view the detailed report.

The meal report may include glucose response analysis presenting the postprandial glucose response metrics calculated from continuous glucose monitoring data. The glucose response analysis may display the peak glucose value, the time to peak, the glucose excursion, the 2-hour glucose value, the area under curve, and the time above threshold. The glucose response analysis may include a graphical representation of the glucose response curve showing glucose levels over time following the meal. The graphical representation may highlight the peak glucose value and may indicate threshold levels for reference.

The meal report may include comparison of the observed glucose response to predicted glucose response. The comparison may indicate whether the actual glucose excursion was larger than predicted, smaller than predicted, or approximately as predicted. The comparison may provide insight into factors that may have contributed to deviations from predicted glucose response, such as eating pace, food composition differences from image analysis estimates, or physiological factors not captured in the prediction model.

The meal report may include comparison of the observed glucose response to the user's historical glucose responses for similar meals. The comparison may indicate whether the current meal produced a glucose response that was better than typical, worse than typical, or approximately typical for meals with similar food properties. The historical comparison may enable the user to track progress over time and to identify meals that consistently produce favorable or unfavorable glucose responses.

The meal report may include personalized recommendations based on the glucose response analysis and eating behavior observed during the meal. The personalized recommendations may suggest modifications for future similar meals, such as reducing portion size, substituting lower glycemic index alternatives, adding fiber or protein to the meal, or eating more slowly. The personalized recommendations may be generated by a machine learning engine 420 based on analysis of factors associated with favorable glucose responses in the user's historical data.

The meal report may include analysis of eating behavior during the meal and the relationship between eating behavior and glucose response. The eating behavior analysis may indicate the average chewing cadence, the deviation from target chewing cadence, the meal duration, and the compliance quality score. The eating behavior analysis may highlight periods during the meal when eating pace deviated substantially from the target pace. The eating behavior analysis may indicate whether the observed eating behavior was associated with the glucose response outcome, such as indicating that rapid eating during the first portion of the meal may have contributed to a larger than expected glucose excursion.

The meal report may include feedback on therapeutic intervention effectiveness. The feedback may indicate the gastric vibrational therapy parameters that were applied during the meal and the post-meal period. The feedback may indicate whether the therapeutic intervention appeared to be effective based on comparison of the observed glucose response to predicted glucose response without intervention. The feedback may inform the user regarding the value of the therapeutic intervention and may reinforce adherence to therapy protocols.

The system may provide haptic alerts through the user interface when severe deviation from target chewing cadence is detected. Haptic alerts may comprise vibration patterns delivered through a mobile device, a wearable device, or another device capable of generating tactile feedback. The haptic alerts may provide immediate notification of eating pace deviation without requiring the user to view a display screen, enabling the user to receive feedback while focused on eating or conversation.

The haptic alerts may be triggered when the calculated deviation between the current chewing cadence and the target chewing cadence exceeds a severe deviation threshold. The severe deviation threshold may be set at a deviation percentage indicating substantial departure from the target eating pace, such as 30 percent, 40 percent, or 50 percent above the target chewing cadence. The severe deviation threshold may be higher than thresholds used for visual feedback or therapeutic parameter adjustment, reserving haptic alerts for conditions warranting immediate user attention.

The haptic alerts may comprise distinct vibration patterns that communicate the nature of the deviation. A first vibration pattern may indicate that the user is eating faster than the target pace and should slow down. A second vibration pattern may indicate that the user is eating substantially faster than the target pace and should slow down immediately. The distinct vibration patterns may enable the user to distinguish between moderate and severe deviations based on the tactile feedback without viewing a display.

The haptic alerts may be delivered through a mobile device held by the user or placed on a table near the user during eating. The mobile device may generate vibration using a vibration motor or haptic actuator integrated into the mobile device. The vibration may be perceptible to the user through contact with the mobile device or through transmission of vibration through the table surface.

The haptic alerts may be delivered through a wearable device worn by the user during eating. The wearable device may comprise a smartwatch, a fitness tracker, or another wrist-worn device capable of generating haptic feedback. The wearable device may comprise a ring-form device worn on a finger. The wearable device may comprise an earbud or other ear-worn device capable of generating haptic feedback. The delivery of haptic alerts through a wearable device may provide discrete notification that is perceptible to the user without being noticeable to dining companions.

The haptic alerts may be configured to repeat at intervals while the severe deviation condition persists. When the calculated deviation exceeds the severe deviation threshold, the system may deliver an initial haptic alert and may deliver subsequent haptic alerts at specified intervals, such as every 30 seconds or every minute, until the deviation falls below the severe deviation threshold. The repeated haptic alerts may provide persistent notification that encourages the user to modify eating pace.

The haptic alerts may be combined with visual alerts displayed on the user interface when severe deviation is detected. The combination of haptic and visual alerts may provide multi-modal notification that increases the likelihood of user awareness and response. The visual alerts may include prominent display elements such as flashing indicators, color changes, or pop-up notifications that complement the haptic feedback.

The user may configure haptic alert preferences through settings in the user interface module 424. The configurable preferences may include enabling or disabling haptic alerts, selecting the severe deviation threshold that triggers haptic alerts, selecting the vibration pattern intensity, and selecting the device through which haptic alerts are delivered. The configurable preferences may enable personalization of the haptic alert functionality based on user preferences and social context considerations.

With reference to FIG. 1, the controller may be implemented on a smartphone with a mobile application 104 serving as a processing component that controls both chewing feedback and gastric vibrational therapy. In this implementation, the smartphone may host the mobile application 104 that receives data from chewing detection sensors and physiological monitoring devices, processes the received data using algorithms executed on the smartphone processor, and generates control signals that are transmitted to the gastric vibrational therapy device. The mobile application 104 may serve as the processing brain that coordinates the various sensing and actuation components of the system. The smartphone implementation may leverage the computational capabilities of modern smartphones, which include processors capable of executing machine learning models, sufficient memory for storing historical meal data, and wireless communication modules for receiving sensor data and transmitting control signals.

The mobile application 104 implemented on the smartphone may provide chewing feedback to the user through the smartphone display, speakers, or haptic actuator. The chewing feedback may include visual displays of current chewing cadence relative to target chewing cadence, auditory cues such as rhythmic tones or verbal prompts to guide chewing pace, and haptic alerts when substantial deviation from target chewing cadence is detected. The mobile application 104 may coordinate the chewing feedback with the gastric vibrational therapy control, adjusting both the user-facing feedback and the therapeutic device parameters based on the detected eating behavior and physiological state.

The mobile application 104 implemented on the smartphone may transmit control signals to the gastric vibrational therapy device via a wireless communication protocol. The control signals may specify operational parameters including vibration frequency, vibration amplitude, duty cycle, and activation timing. The gastric vibrational therapy device may receive the control signals through a communication module and adjust operation of a vibration actuator according to the received parameters. The smartphone-based implementation may enable the mobile application 104 to serve as a central controller that coordinates sensing, processing, and actuation across multiple distributed components.

In an alternative implementation, the controller may be implemented within the gastric vibrational therapy device 416 itself with internal artificial intelligence (AI) functions to process data and control vibration. In this implementation, the gastric vibrational therapy device 416 may include a processor, memory, and software configured to execute machine learning models and control algorithms without requiring an external smartphone or mobile application for processing. The gastric vibrational therapy device 416 with internal AI functions may receive sensor data directly from chewing detection sensors and physiological monitoring devices, process the received data using onboard computational resources, and control the vibration actuator 502 based on the processing results.

With reference to FIG. 5, the gastric vibrational therapy device 416 with internal AI capabilities may include a microcontroller or microprocessor with sufficient computational power to execute machine learning inference operations. The microcontroller or microprocessor may be integrated into control electronics 504 within the gastric vibrational therapy device 416 housing. The gastric vibrational therapy device 416 may include memory configured to store machine learning model parameters, historical meal data, and user-specific settings. The onboard memory may enable the gastric vibrational therapy device 416 to maintain personalized models and learned patterns without requiring continuous connection to an external device.

The gastric vibrational therapy device 416 with internal AI functions may receive chewing data directly from a chewing detection sensor 406 via a wireless communication protocol. The gastric vibrational therapy device 416 may process the received chewing data to calculate chewing cadence and detect deviations from target chewing cadence. The gastric vibrational therapy device 416 may receive physiological data directly from physiological monitoring devices and integrate the physiological data with the chewing data to determine appropriate therapeutic parameters. The internal AI functions may enable the gastric vibrational therapy device 416 to perform the complete processing pipeline from sensor data acquisition through therapeutic parameter determination without external processing support.

The gastric vibrational therapy device 416 with internal AI functions may operate autonomously during eating episodes without requiring a smartphone to be present or active. The autonomous operation may provide convenience for users who prefer not to interact with a smartphone during meals. The autonomous operation may provide reliability by eliminating dependence on smartphone battery status, wireless connectivity, or application availability. The gastric vibrational therapy device 416 may synchronize data with a smartphone application when connectivity is available, enabling the smartphone application to provide user interface functions, data visualization, and cloud backup while the gastric vibrational therapy device 416 handles real-time sensing and control.

The gastric vibrational therapy device 416 with internal AI functions may include edge computing that enables execution of machine learning models on the device. Edge computing refers to computational processing performed on devices at the periphery of a network rather than in centralized cloud servers. The edge computing may enable the gastric vibrational therapy device 416 to perform machine learning inference with low latency, as the processing occurs locally without requiring data transmission to and from external servers. The low latency processing may enable responsive real-time control of therapeutic parameters based on detected eating behavior.

The mobile application 104 may include a silent mode feature enabling low-noise operation of the gastric vibrational therapy device 416. The silent mode feature may be accessible through a user interface of the mobile application 104, enabling the user to activate silent mode when discrete operation is desired. Silent mode may be appropriate for social dining situations, professional settings, or other contexts where audible vibration noise from the gastric vibrational therapy device 416 may be undesirable or socially awkward.

When silent mode is activated, the mobile application 104 may transmit control signals to the gastric vibrational therapy device 416 that adjust operational parameters to minimize audible noise generation. The operational parameter adjustments for silent mode may include reducing vibration amplitude to a level that produces therapeutic effect while generating minimal audible noise. The operational parameter adjustments may include selecting vibration frequencies that are less audible to human hearing while maintaining therapeutic effectiveness. The operational parameter adjustments may include modifying duty cycle patterns to reduce the perceptibility of vibration sounds.

The silent mode operation may involve reducing vibration amplitude to a percentage of the standard amplitude, such as 50 percent or 60 percent of the amplitude that would otherwise be applied based on the risk assessment. The reduced amplitude may decrease the mechanical energy transmitted through the gastric vibrational therapy device 416 housing and attachment mechanism, thereby reducing the acoustic energy radiated as audible sound. The reduced amplitude in silent mode may provide a trade-off between therapeutic intensity and operational discretion, with the user accepting potentially reduced therapeutic effect in exchange for quiet operation.

The silent mode operation may involve selecting vibration frequencies in ranges that are less perceptible to human hearing. Human auditory sensitivity varies across the frequency spectrum, with reduced sensitivity at very low frequencies and very high frequencies compared to mid-range frequencies. The silent mode may select vibration frequencies at the lower end of the operational frequency range, such as frequencies below 100 Hz, where the vibration may be felt by the user but may generate less audible sound compared to higher frequencies. The frequency selection for silent mode may be constrained by the requirement to maintain therapeutic effectiveness, as certain frequency ranges may be associated with enhanced vagal activation or mechanoreceptor stimulation.

The silent mode operation may involve modifying the vibration pattern to reduce acoustic perceptibility. Continuous vibration may generate a sustained audible hum, while pulsed vibration may generate intermittent sounds that may be more noticeable due to the onset and offset transients. The silent mode may employ gradual ramping of vibration amplitude at the beginning and end of each pulse to reduce the sharpness of onset and offset transients. The gradual ramping may produce a smoother acoustic profile that is less attention-capturing than abrupt vibration onset.

The mobile application 104 may provide automatic silent mode activation based on detected context. The mobile application 104 may detect social dining context through location data, calendar data, or audio analysis indicating the presence of multiple people. When social dining context is detected, the mobile application 104 may automatically activate silent mode without requiring manual user input. The automatic activation may ensure discrete operation in social situations even when the user forgets to manually activate silent mode.

The mobile application 104 may provide scheduled silent mode activation based on user-configured time periods. The user may configure time periods during which silent mode is automatically activated, such as during work hours or during evening hours when the user typically dines with family. The scheduled activation may provide consistent discrete operation during recurring time periods without requiring manual activation for each eating episode.

The silent mode feature may be implemented in conjunction with the gastric vibrational therapy device 416 with internal AI functions. When the gastric vibrational therapy device 416 includes internal processing functions, the silent mode settings may be stored in onboard memory and applied by the internal controller without requiring real-time communication with the mobile application 104. The gastric vibrational therapy device 416 may receive silent mode configuration from the mobile application 104 during synchronization and apply the silent mode parameters autonomously during subsequent eating episodes.

The gastric vibrational therapy device 416 may include soundproofing features that complement the silent mode operational parameter adjustments. The soundproofing features may include acoustic insulation materials positioned between the vibration actuator 502 and the device housing to reduce transmission of vibration-induced sound to the external environment. The soundproofing features may include vibration dampening structures that absorb mechanical energy and reduce radiation of acoustic energy from the device housing. The combination of silent mode operational parameter adjustments and physical soundproofing features may provide enhanced noise reduction compared to either approach alone.

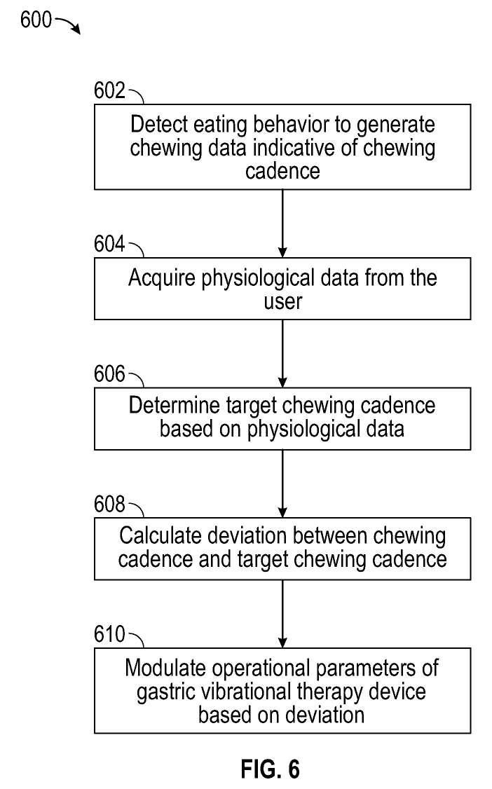

FIG. 6 shows a method 600 for adaptive control of gastric satiety therapy, in accordance with one exemplary embodiment of the present disclosure. The order in which the method 600 is described should not be construed as a limitation, and any number of the described process blocks can be combined in any order to implement the process or alternate processes. Additionally, individual blocks may be deleted from the method 600 without departing from the spirit and scope of the invention described herein. Furthermore, the method 600 can be implemented in any suitable hardware, software, firmware, or combination thereof. The method 600 may be performed by a controller 418 in communication with a chewing detection sensor 406, a physiological monitoring device, and a gastric vibrational therapy device 416.

The method 600 may start at step 602. At step 602, the chewing detection sensor 406 may detect eating behavior of a user to generate chewing data indicative of a chewing cadence. The chewing detection sensor 406 may monitor the user during an eating episode to capture real-time eating behavior information. The chewing detection sensor 406 may comprise an acoustic sensor, an inertial measurement unit, an electromyographic sensor, or a strain sensor configured to detect chewing activity. The chewing data generated at step 602 may include detected chewing events, calculated chewing cadence values expressed in chews per minute, inter-chew intervals, and swallow timing information. The chewing detection sensor 406 may process sensor signals to identify chewing events and calculate the chewing cadence over a sliding time window during the eating episode. The chewing detection sensor 406 may detect a chewing cadence of 45 chews per minute for a user consuming a meal, with inter-chew intervals averaging 1.33 seconds between successive chewing events.

At step 604, the controller 418 may acquire physiological data from the user using the physiological monitoring device 407. The controller 418 may receive data from the physiological monitoring device 407 configured to measure physiological parameters indicative of the user's metabolic state. The physiological monitoring device 407 may comprise the continuous glucose monitor 408 configured to measure glucose levels, the heart rate variability monitor 410 configured to measure heart rate variability metrics, the sleep tracking device 412 configured to monitor sleep quality, or the activity tracker 414 configured to monitor physical activity. The physiological data acquired at step 604 may include current glucose level, glucose rate-of-change, glucose trend direction, heart rate variability metrics such as RMSSD and LF/HF ratio, sleep duration, sleep quality scores, and activity level data. The controller 418 may receive the physiological data wirelessly from the physiological monitoring device 407 via a communication protocol such as Bluetooth. The controller 418 may receive physiological data indicating a current glucose level of 125 mg/dL with a rising trend, an RMSSD value of 28 milliseconds indicating reduced vagal tone, and a sleep quality score of 62% from the preceding night.

At step 606, the controller 418 may determine a target chewing cadence based on the physiological data. The controller 418 may analyze the physiological data acquired at step 604 to establish an appropriate target chewing cadence tailored to the user's current physiological condition. The controller 418 may apply algorithms that map the physiological data to a target chewing cadence value expressed in chews per minute. When the physiological data indicates elevated baseline glucose levels, the controller 418 may determine a lower target chewing cadence to promote slower eating. When the physiological data indicates low vagal tone as reflected by reduced heart rate variability metrics, the controller 418 may determine a lower target chewing cadence to encourage a calmer eating pace. When the physiological data indicates poor sleep quality from the preceding night, the controller 418 may determine a lower target chewing cadence to compensate for metabolic effects of sleep deprivation. The controller 418 may determine a target chewing cadence of 32 chews per minute based on the elevated glucose level of 125 mg/dL, the reduced RMSSD value of 28 milliseconds, and the sleep quality score of 62%. The target chewing cadence determined at step 606 may represent an eating pace that is appropriate for the user's current physiological state.

At step 608, the controller 418 may calculate a deviation between the chewing cadence and the target chewing cadence. The controller 418 may compare the actual chewing cadence detected at step 602 with the target chewing cadence determined at step 606 to quantify any difference between the two values. The deviation may be calculated as the difference between the chewing cadence and the target chewing cadence. A positive deviation may indicate that the user is chewing faster than the target chewing cadence. A negative deviation may indicate that the user is chewing slower than the target chewing cadence. The deviation may be calculated as an absolute difference expressed in chews per minute or as a percentage difference calculated by dividing the absolute difference by the target chewing cadence. The controller 418 may calculate a deviation of 13 chews per minute, representing a 40.6% positive deviation indicating that the user's actual chewing cadence of 45 chews per minute exceeds the target chewing cadence of 32 chews per minute. The controller 418 may calculate the deviation at regular intervals throughout the eating episode using a sliding window approach that provides smoothed deviation values.

At step 610, the controller 418 may modulate operational parameters of the gastric vibrational therapy device 416 based on the calculated deviation. The controller 418 may adjust parameters such as vibration frequency, vibration amplitude, duty cycle, or stimulation duration of the gastric vibrational therapy device 416 to provide adaptive therapeutic intervention in response to the detected eating behavior. The gastric vibrational therapy device 416 may deliver non-invasive mechanical stimulation to a gastric region of the user based on the modulated operational parameters. When the calculated deviation indicates that the chewing cadence exceeds the target chewing cadence, the controller 418 may increase the vibration amplitude to provide enhanced mechanical stimulation that compensates for rapid eating behavior. When the calculated deviation indicates that the chewing cadence is at or below the target chewing cadence, the controller 418 may maintain or decrease the vibration amplitude. The controller 418 may increase the vibration amplitude from 0.6 g-force to 0.95 g-force based on the 40.6% positive deviation detected at step 608. The magnitude of the operational parameter adjustment may be proportional to the magnitude of the calculated deviation, enabling graduated therapeutic response. The modulation of operational parameters at step 610 may enable the gastric vibrational therapy device 416 to provide real-time therapeutic intervention that responds to eating behavior as the eating episode progresses.

The method 600 may implement closed-loop control wherein steps 602, 604, 606, 608, and 610 may be performed repeatedly throughout the eating episode. The method 600 may return to step 602 following step 610 to continue the continuous monitoring cycle, with the chewing detection sensor 406 detecting additional chewing events for subsequent analysis by the controller 418. The closed-loop control may enable the controller 418 to continuously monitor eating behavior, update the target chewing cadence based on changing physiological conditions, recalculate the deviation, and adjust the operational parameters of the gastric vibrational therapy device 416 in response to the detected eating behavior. The continuous execution of the method 600 during the eating episode may provide adaptive therapeutic intervention that tracks the user's eating behavior in real-time and adjusts the gastric vibrational therapy accordingly.

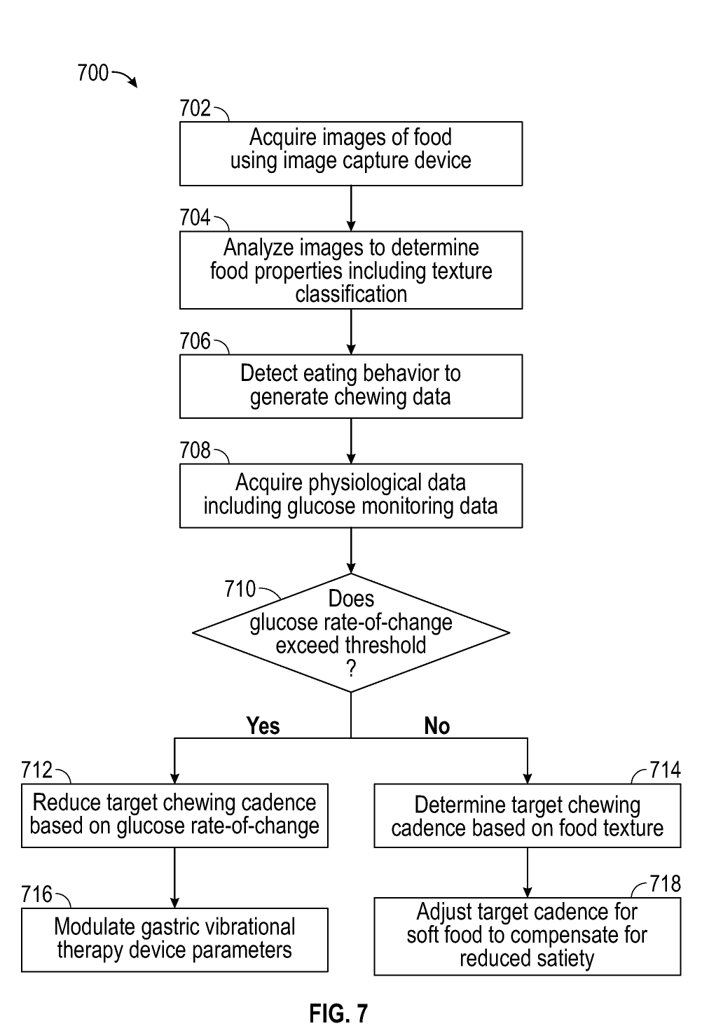

FIG. 7 shows a method 700 for adaptive control of gastric satiety therapy with food image analysis and glucose monitoring, in accordance with one exemplary embodiment of the present invention. The order in which the method 700 is described should not be construed as a limitation, and any number of the described process blocks can be combined in any order to implement the process or alternate processes. Additionally, individual blocks may be deleted from the method 700 without departing from the spirit and scope of the invention described herein. Furthermore, the method 700 can be implemented in any suitable hardware, software, firmware, or combination thereof. The method 700 may be performed by a controller 418 in communication with an image capture device 402, an image analysis module 404, a chewing detection sensor 406, a continuous glucose monitor 408, and a gastric vibrational therapy device 416.

The method 700 may start at step 702. At step 702, the image capture device 402 may acquire images of food before consumption by the user. The image capture device 402 may comprise a camera integrated into a smartphone, a tablet, a dedicated device, or a wearable device such as smart glasses. The user may photograph food from an overhead angle to facilitate accurate portion size estimation. The image capture device 402 may provide guidance to the user regarding image capture, including prompts for reference object placement and lighting optimization. The acquired images may be stored in memory 422 and transmitted to the image analysis module 404 for processing. The image capture device 402 may capture an overhead image of a plate containing pasta with marinara sauce and a side salad, with a standard-sized fork visible in the frame as a reference object for portion size estimation.

At step 704, the image analysis module 404 may analyze the acquired images to determine food properties including a food texture classification. The image analysis module 404 may process the food images acquired at step 702 using convolutional neural networks trained on food image databases to identify foods present in the acquired images. The food properties determined at step 704 may include food identification, texture classification, portion size estimation, macronutrient composition, estimated caloric content, and glycemic index prediction. The image analysis module 404 may categorize the identified foods into texture categories including very soft, soft, moderate, firm, and very firm based on texture features extracted from the food images. The texture classification may indicate the chewing requirement associated with the identified foods, with soft foods requiring less chewing effort and firm foods requiring more chewing effort. The image analysis module 404 may identify the pasta as a soft texture food with an estimated caloric content of 450 calories, a macronutrient composition of 65% carbohydrates, 15% protein, and 20% fat, and a glycemic index prediction of 55.

At step 706, the chewing detection sensor 406 may detect eating behavior of the user to generate chewing data indicative of a chewing cadence. The chewing detection sensor 406 may monitor the user during an eating episode to capture real-time eating behavior information. The chewing detection sensor 406 may comprise an acoustic sensor, an inertial measurement unit, an electromyographic sensor, or a strain sensor configured to detect chewing activity. The chewing data generated at step 706 may include detected chewing events, calculated chewing cadence values expressed in chews per minute, inter-chew intervals, and swallow timing information. The chewing detection sensor 406 may process sensor signals to identify chewing events and calculate the chewing cadence over a sliding time window during the eating episode. The chewing detection sensor 406 may detect a chewing cadence of 42 chews per minute for the user consuming the pasta meal, with inter-chew intervals averaging 1.43 seconds between successive chewing events.

At step 708, the physiological monitoring device 407 may acquire physiological data including glucose monitoring data from the user. The continuous glucose monitor 408 may measure interstitial glucose levels continuously and provide real-time glucose data including a current glucose level, a glucose trend direction, and a glucose rate-of-change metric. The glucose rate-of-change metric may represent the speed at which glucose levels are changing over time, expressed in milligrams per deciliter per minute (mg/dL/min). The controller 418 may also receive physiological data from the heart rate variability monitor 410, the sleep tracking device 412, and the activity tracker 414. The physiological data may be received wirelessly from the physiological monitoring device 407 via a communication protocol such as Bluetooth through the communication module 426. The continuous glucose monitor 408 may provide physiological data indicating a current glucose level of 118 mg/dL with a rising trend and a glucose rate-of-change metric of 2.5 mg/dL/min.

At step 710, the controller 418 may determine whether the glucose rate-of-change exceeds a threshold. The controller 418 may compare the glucose rate-of-change metric acquired at step 708 to a threshold value indicating rapid glucose elevation. The threshold may be set at a value such as 2 mg/dL/min, which may indicate rapidly rising glucose levels that warrant intervention. If the glucose rate-of-change exceeds the threshold at step 710, the method 700 may proceed to step 712. If the glucose rate-of-change does not exceed the threshold at step 710, the method 700 may proceed to step 714.

If the glucose rate-of-change exceeds the threshold at step 710, the method 700 may proceed to step 712. At step 712, the controller 418 may reduce the target chewing cadence based on the glucose rate-of-change. The controller 418 may decrease the target chewing cadence to encourage slower eating that may help moderate the glucose response. The reduction of the target chewing cadence when the glucose rate-of-change exceeds the threshold may promote slower eating during periods of rapid glucose elevation. Slower eating may extend the duration of food consumption, which may distribute carbohydrate absorption over a longer time period and reduce the rate of glucose elevation. The magnitude of the target chewing cadence reduction may be proportional to the magnitude by which the glucose rate-of-change metric exceeds the threshold. The controller 418 may reduce the target chewing cadence from a baseline of 35 chews per minute to 28 chews per minute based on the glucose rate-of-change metric of 2.5 mg/dL/min exceeding the threshold of 2 mg/dL/min. From step 712, the method 700 may continue to step 716.

If the glucose rate-of-change does not exceed the threshold at step 710, the method 700 may proceed to step 714. At step 714, the controller 418 may determine the target chewing cadence based on food texture. The controller 418 may adjust the target chewing cadence based on the food texture classification determined at step 704. The target chewing cadence may be inversely proportional to food texture firmness. Soft foods may be assigned a lower target chewing cadence to compensate for reduced mechanical satiety stimulation, while firm foods may be assigned a higher target chewing cadence as the texture naturally slows eating pace. The texture-responsive target chewing cadence determination may enable the method 700 to adjust eating pace targets based on the satiety-promoting characteristics of the food being consumed. The controller 418 may determine a target chewing cadence of 30 chews per minute based on the soft texture classification of the pasta identified at step 704. From step 714, the method 700 may continue to step 718.

At step 718, the controller 418 may adjust the target cadence for soft food to compensate for reduced satiety. The controller 418 may decrease the target chewing cadence when the food texture classification indicates a soft food texture. Soft foods may require less chewing effort and may be associated with reduced mechanical satiety signaling compared to firmer foods. By decreasing the target chewing cadence for soft foods, the method 700 may encourage slower eating that compensates for the reduced mechanical satiety stimulation provided by soft food textures. The controller 418 may apply a texture adjustment factor to the target chewing cadence, with soft food textures receiving adjustment factors less than 1.0 that reduce the target chewing cadence. The controller 418 may apply a texture adjustment factor of 0.85 to the target chewing cadence, reducing the target from 35 chews per minute to 30 chews per minute for the soft pasta texture. A very soft food texture may result in a target chewing cadence that is 70 percent of a baseline target chewing cadence, while a soft food texture may result in a target chewing cadence that is 85 percent of the baseline target chewing cadence.

At step 716, the controller 418 may modulate operational parameters of the gastric vibrational therapy device 416 based on the target chewing cadence determined through either the glucose-responsive pathway (via step 712) or the texture-responsive pathway (via steps 714 and 718). The operational parameters modulated at step 716 may include vibration frequency, vibration amplitude, duty cycle, and stimulation duration. The controller 418 may generate control signals that adjust the operational parameters based on the calculated deviation between the actual chewing cadence detected at step 706 and the target chewing cadence determined through the applicable pathway. When the calculated deviation indicates that the chewing cadence exceeds the target chewing cadence, the controller 418 may increase the vibration amplitude to provide increased mechanical stimulation. The controller 418 may calculate a deviation of 14 chews per minute between the actual chewing cadence of 42 chews per minute and the reduced target chewing cadence of 28 chews per minute, representing a 50% positive deviation. The controller 418 may increase the vibration amplitude of the gastric vibrational therapy device 416 from 0.5 g-force to 0.85 g-force based on the calculated deviation. The gastric vibrational therapy device 416 may deliver non-invasive mechanical stimulation to a gastric region of the user based on the modulated operational parameters through the vibration actuator 502.

The method 700 may provide an adaptive approach that integrates food image analysis with physiological monitoring to determine appropriate chewing cadence targets and therapy parameters. The decision point at step 710 may enable the method 700 to respond to glucose dynamics by adjusting the target chewing cadence when elevated glucose rate-of-change is detected. The parallel pathways following the decision point may allow the method 700 to address different conditions through either glucose-responsive cadence reduction or texture-based cadence adjustment for soft foods. The integration of food texture classification with glucose monitoring may enable the method 700 to provide personalized therapeutic intervention that accounts for both the characteristics of the food being consumed and the real-time metabolic response of the user. The method 700 may return to step 702 following step 716 to continue the continuous monitoring cycle, with the image capture device 402 acquiring additional food images and the chewing detection sensor 406 detecting subsequent eating behavior for analysis by the controller 418.

In the context of a method for adaptive control of gastric satiety therapy, the method 700 may further comprise acquiring images of food using an image capture device 402 as performed at step 702, analyzing the acquired images to determine food properties including a food texture classification as performed at step 704, and adjusting the target chewing cadence based on the food texture classification as performed at steps 714 and 718. The food texture classification may categorize foods into texture categories based on chewing requirements, with soft foods requiring less chewing effort and firm foods requiring more chewing effort. The adjustment of the target chewing cadence based on the food texture classification may enable the method 700 to provide eating pace guidance that is appropriate for the specific food being consumed.

In the context of a method for adaptive control of gastric satiety therapy, adjusting the target chewing cadence may comprise decreasing the target chewing cadence when the food texture classification indicates a soft food texture to compensate for reduced mechanical satiety stimulation. Soft foods may provide less mechanical stimulation to gastric mechanoreceptors during chewing compared to firmer foods. The reduced mechanical satiety stimulation from soft foods may result in diminished satiety signaling through vagal afferent pathways. By decreasing the target chewing cadence when soft food texture is detected, the method 700 may encourage slower eating that extends the duration of food consumption and provides additional time for satiety signals to develop. The decreased target chewing cadence for soft foods may compensate for the reduced mechanical satiety stimulation by promoting an eating pace that enhances other satiety signaling mechanisms including hormonal responses and gastric distension signaling.

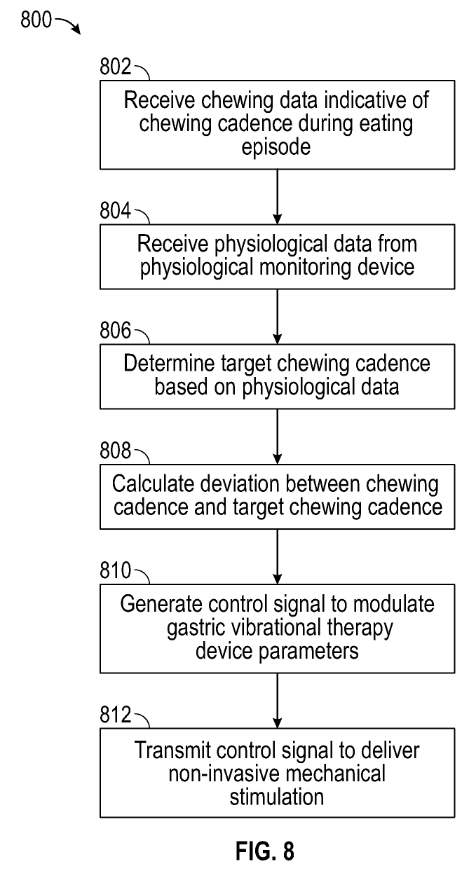

FIG. 8 shows a method 800 for adaptive control of gastric satiety therapy, in accordance with one embodiment of the present invention. The order in which the method 800 is described should not be construed as a limitation, and any number of the described process blocks can be combined in any order to implement the process or alternate processes. Additionally, individual blocks may be deleted from the method 800 without departing from the spirit and scope of the invention described herein. Furthermore, the method 800 can be implemented in any suitable hardware, software, firmware, or combination thereof.

The method 800 may start at step 802. At step 802, the chewing detection sensor 406 may generate chewing data indicative of a chewing cadence during an eating episode. The chewing detection sensor 406 may comprise an acoustic sensor, an inertial measurement unit, an electromyographic sensor, or a strain sensor configured to detect chewing activity. The chewing detection sensor 406 may transmit the chewing data to the controller 418 via a wireless communication protocol such as Bluetooth or via a wired connection. The chewing data may include raw sensor signals, detected chewing events, calculated chewing cadence values expressed in chews per minute, inter-chew intervals, and swallow timing information. The controller 418 may receive the chewing data in real-time as the user consumes food, enabling the controller 418 to monitor eating behavior throughout the eating episode.

Further, the chewing detection sensor 406 may detect a chewing cadence of 48 chews per minute for a user consuming a meal, with inter-chew intervals averaging 1.25 seconds between successive chewing events. The chewing detection sensor 406 may process acoustic signals using band-pass filtering and peak detection to identify individual chewing events and may calculate the chewing cadence over a 30-second sliding time window.

At step 804, the controller 418 may receive physiological data from the physiological monitoring device 407. The physiological monitoring device 407 may comprise the continuous glucose monitor 408 configured to measure glucose levels, the heart rate variability monitor 410 configured to measure heart rate variability metrics, the sleep tracking device 412 configured to monitor sleep quality, or the activity tracker 414 configured to monitor physical activity. The physiological data received at step 804 may include current glucose level, glucose rate-of-change values, glucose trend direction, heart rate variability metrics such as RMSSD and LF/HF ratio, sleep duration, sleep quality scores, and activity level data. The controller 418 may receive the physiological data via wireless communication protocols through the communication module 426 or through application programming interfaces that provide access to data stored by the physiological monitoring device 407.

Further, the continuous glucose monitor 408 of the physiological monitoring device 407 may provide physiological data indicating a current glucose level of 132 mg/dL with a rising trend and a glucose rate-of-change of 2.1 mg/dL/min. The heart rate variability monitor 410 of the physiological monitoring device 407 may provide an RMSSD value of 31 milliseconds indicating moderate vagal tone. The sleep tracking device 412 may provide a sleep quality score of 58% from the preceding night, indicating suboptimal sleep that may affect metabolic responses.

At step 806, the controller 418 may determine a target chewing cadence based on the physiological data. The controller 418 may analyze the physiological data received at step 804 to establish an appropriate target chewing cadence tailored to the user's current physiological condition. The controller 418 may apply algorithms that map the physiological data to a target chewing cadence value expressed in chews per minute. When the physiological data indicates elevated baseline glucose levels, the controller 418 may determine a lower target chewing cadence to promote slower eating that may reduce postprandial glucose excursions. When the physiological data indicates low vagal tone as reflected by reduced heart rate variability metrics, the controller 418 may determine a lower target chewing cadence to encourage a calmer eating pace that may enhance parasympathetic activation. When the physiological data indicates poor sleep quality from the preceding night, the controller 418 may determine a lower target chewing cadence to compensate for metabolic effects of sleep deprivation.

Further, the controller 418 may determine a target chewing cadence of 30 chews per minute based on the elevated glucose level of 132 mg/dL, the moderate RMSSD value of 31 milliseconds, and the sleep quality score of 58%. The controller 418 may apply a glucose-based adjustment factor of 1.15 due to the elevated baseline glucose and a sleep-based adjustment factor of 1.10 due to the suboptimal sleep quality, resulting in a reduced target chewing cadence compared to baseline parameters.

At step 808, the controller 418 may calculate a deviation between the chewing cadence and the target chewing cadence. The controller 418 may compare the actual chewing cadence indicated by the chewing data received at step 802 with the target chewing cadence determined at step 806 to quantify the difference between the two values. The deviation may be calculated as the difference between the chewing cadence and the target chewing cadence. A positive deviation may indicate that the user is chewing faster than the target chewing cadence. A negative deviation may indicate that the user is chewing slower than the target chewing cadence. A deviation near zero may indicate that the user is chewing at approximately the target chewing cadence. The deviation may be calculated as an absolute difference expressed in chews per minute or as a percentage difference calculated by dividing the absolute difference by the target chewing cadence and multiplying by 100.

Further, the controller 418 may calculate a deviation of 18 chews per minute between the actual chewing cadence of 48 chews per minute and the target chewing cadence of 30 chews per minute, representing a 60% positive deviation indicating that the user is eating substantially faster than the target pace.

At step 810, the controller 418 may generate a control signal to modulate operational parameters of the gastric vibrational therapy device 416 based on the calculated deviation. The controller 418 may generate a control signal that specifies adjustments to operational parameters of the gastric vibrational therapy device 416 in response to the deviation calculated at step 808. The operational parameters may include vibration frequency, vibration amplitude, duty cycle, and stimulation duration. When the calculated deviation indicates that the chewing cadence exceeds the target chewing cadence, the controller 418 may generate a control signal that increases the vibration amplitude to provide enhanced mechanical stimulation that compensates for rapid eating behavior. When the calculated deviation indicates that the chewing cadence is at or below the target chewing cadence, the controller 418 may generate a control signal that maintains or decreases the vibration amplitude. The magnitude of the operational parameter adjustment specified in the control signal may be proportional to the magnitude of the calculated deviation, enabling graduated therapeutic response across varying degrees of deviation from target eating behavior.

Further, the controller 418 may generate a control signal specifying an increase in vibration amplitude from 0.5 g-force to 0.9 g-force based on the 60% positive deviation detected at step 808. The control signal may also specify an increase in duty cycle from 70% to 85% to provide more sustained mechanical stimulation during the eating episode.

At step 812, the controller 418 may transmit the control signal to the gastric vibrational therapy device 416 to deliver non-invasive mechanical stimulation to a gastric region of the user. The controller 418 may transmit the control signal via the communication module 426 using a wireless communication protocol such as Bluetooth. The gastric vibrational therapy device 416 may receive the control signal through the GVT communication module 510 and the control electronics 504 may adjust operation of the vibration actuator 502 according to the operational parameters specified in the control signal. The vibration actuator 502 may deliver non-invasive mechanical stimulation to the gastric region based on the modulated operational parameters, providing therapeutic intervention that responds to the eating behavior detected during the eating episode.

Further, the gastric vibrational therapy device 416 may receive the control signal and the vibration actuator 502 may increase vibration amplitude to 0.9 g-force at a frequency of 120 Hz with an 85% duty cycle. The sensors 508 within the gastric vibrational therapy device 416 may verify that the vibration actuator 502 is operating at the intended parameters and may monitor device temperature to ensure safe operation.

The method 800 may return to step 802 following step 812 to continue the continuous monitoring cycle, with the chewing detection sensor 406 generating additional chewing data for subsequent analysis by the controller 418. The steps 802, 804, 806, 808, 810, and 812 may be performed repeatedly throughout the eating episode to provide continuous adaptive control. The controller 418 may continuously receive updated chewing data and physiological data, recalculate the target chewing cadence and deviation, generate updated control signals, and transmit the updated control signals to the gastric vibrational therapy device 416.

In the context of a non-transitory computer-readable medium storing instructions, the instructions, when executed by a processor, may cause the processor to receive chewing data from a chewing detection sensor, the chewing data indicative of a chewing cadence of a user during an eating episode as performed at step 802. The instructions may cause the processor to receive physiological data from a physiological monitoring device as performed at step 804. The instructions may cause the processor to determine a target chewing cadence based on the physiological data as performed at step 806. The instructions may cause the processor to calculate a deviation between the chewing cadence and the target chewing cadence as performed at step 808. The instructions may cause the processor to generate a control signal to modulate operational parameters of a gastric vibrational therapy device based on the calculated deviation as performed at step 810. The instructions may cause the processor to transmit the control signal to the gastric vibrational therapy device to deliver non-invasive mechanical stimulation to a gastric region of the user as performed at step 812.

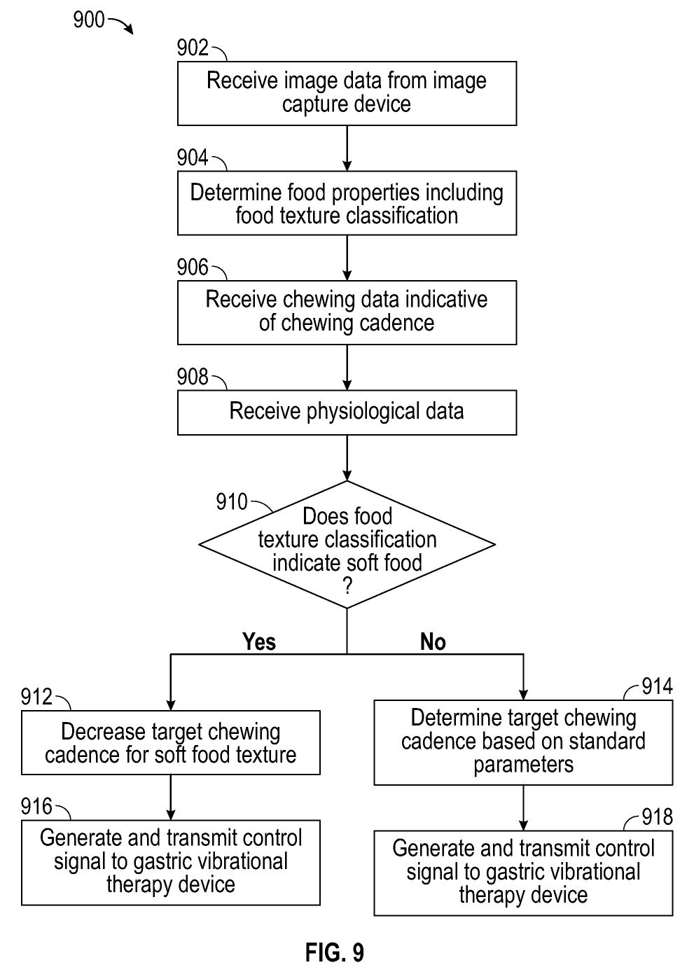

FIG. 9 shows a method 900 for adaptive control of gastric satiety therapy with food texture-based target chewing cadence adjustment, in accordance with one embodiment of the present invention. The order in which the method 900 is described should not be construed as a limitation, and any number of the described process blocks can be combined in any order to implement the process or alternate processes. Additionally, individual blocks may be deleted from the method 900 without departing from the spirit and scope of the invention described herein. Furthermore, the method 900 can be implemented in any suitable hardware, software, firmware, or combination thereof.

The method 900 may start at step 902. At step 902, the image capture device 402 may acquire image data of food before consumption by a user. The image capture device 402 may comprise a camera integrated into a smartphone, a tablet, a dedicated device, or a wearable device such as smart glasses. The image capture device 402 may transmit the image data to the controller 418 via a wired connection or a wireless communication protocol through the communication module 426. The image data may comprise one or more digital images depicting food items that the user intends to consume. The image data may include metadata such as timestamp information and image resolution parameters.

Further, the image capture device 402 may capture an overhead image of a plate containing mashed potatoes, steamed vegetables, and grilled chicken, with a standard-sized fork visible in the frame as a reference object for portion size estimation.

At step 904, the image analysis module 404 may determine food properties including a food texture classification from the received image data. The image analysis module 404 may process the image data received at step 902 to extract food properties using convolutional neural networks trained on food image databases. The food properties determined at step 904 may include food identification, texture classification, portion size estimation, macronutrient composition, estimated caloric content, and glycemic index prediction. The image analysis module 404 may categorize the identified foods into texture categories including very soft, soft, moderate, firm, and very firm based on texture features extracted from the image data. The texture classification may indicate the chewing requirement associated with the identified foods, with soft foods requiring less chewing effort and firm foods requiring more chewing effort.

Further, the image analysis module 404 may identify the mashed potatoes as a very soft texture food, the steamed vegetables as a soft texture food, and the grilled chicken as a moderate texture food. The image analysis module 404 may determine an overall meal texture classification of soft based on the predominance of soft-textured foods in the meal, with an estimated caloric content of 520 calories and a macronutrient composition of 45% carbohydrates, 30% protein, and 25% fat.

At step 906, the chewing detection sensor 406 may generate chewing data indicative of a chewing cadence. The chewing detection sensor 406 may monitor eating behavior of the user during an eating episode and may transmit the chewing data to the controller 418. The chewing data may be indicative of a chewing cadence of the user expressed in chews per minute. The chewing data may be received via a wireless communication protocol such as Bluetooth through the communication module 426. The chewing data may include detected chewing events, calculated chewing cadence values, inter-chew intervals, and swallow timing information. The controller 418 may receive the chewing data in real-time as the user consumes food, enabling the controller 418 to monitor eating behavior throughout the eating episode.

Further, the chewing detection sensor 406 may detect a chewing cadence of 38 chews per minute for the user consuming the soft-textured meal, with inter-chew intervals averaging 1.58 seconds between successive chewing events.

At step 908, the controller 418 may receive physiological data from the physiological monitoring device 407. The physiological data received at step 908 may include glucose levels from the continuous glucose monitor 408, glucose rate-of-change values, heart rate variability metrics from the heart rate variability monitor 410, sleep quality data from the sleep tracking device 412, and activity level data from the activity tracker 414. The controller 418 may receive the physiological data via wireless communication protocols through the communication module 426. The physiological data may inform the determination of target chewing cadence in conjunction with the food properties determined at step 904.

Further, the continuous glucose monitor 408 of the physiological monitoring device 407 may provide physiological data indicating a current glucose level of 105 mg/dL with a stable trend. The heart rate variability monitor 410 of the physiological monitoring device 407 may provide an RMSSD value of 42 milliseconds indicating healthy vagal tone. The sleep tracking device 412 may provide a sleep quality score of 78% from the preceding night.

At step 910, the controller 418 may determine whether the food texture classification indicates soft food. The controller 418 may compare the food texture classification determined at step 904 to criteria that define soft food textures. The soft food texture criteria may include the very soft texture category and the soft texture category. Foods classified as very soft may include smoothies, yogurt, pudding, mashed potatoes, and pureed soups. Foods classified as soft may include pasta, white rice, oatmeal, ripe bananas, and cooked vegetables. If the food texture classification indicates soft food, the method 900 may proceed to step 912. If the food texture classification does not indicate soft food, the method 900 may proceed to step 914.

If the food texture classification indicates soft food at step 910, the method 900 may proceed to step 912. At step 912, the controller 418 may decrease the target chewing cadence for soft food texture to compensate for reduced mechanical satiety stimulation. Soft foods may require less chewing effort and may be associated with reduced mechanical satiety signaling compared to firmer foods. The reduced mechanical satiety stimulation from soft foods may result in diminished satiety signaling through vagal afferent pathways. By decreasing the target chewing cadence when soft food texture is detected, the controller 418 may encourage slower eating that extends the duration of food consumption and provides additional time for satiety signals to develop. The decreased target chewing cadence for soft foods may compensate for the reduced mechanical satiety stimulation by promoting an eating pace that enhances other satiety signaling mechanisms including hormonal responses and gastric distension signaling. The magnitude of the target chewing cadence decrease may be proportional to the softness of the food texture, with very soft foods receiving a larger decrease than soft foods.

Further, the controller 418 may apply a texture adjustment factor of 0.85 to a baseline target chewing cadence of 35 chews per minute, resulting in a decreased target chewing cadence of 30 chews per minute for the soft-textured meal. For a very soft food texture, the controller 418 may apply a texture adjustment factor of 0.70, resulting in a target chewing cadence that is 70 percent of the baseline target chewing cadence. From step 912, the method 900 may proceed to step 916.

If the food texture classification does not indicate soft food at step 910, the method 900 may proceed to step 914. At step 914, the controller 418 may determine the target chewing cadence based on standard parameters. The controller 418 may determine the target chewing cadence using standard parameters that do not include the soft food texture adjustment applied at step 912. The standard parameters may include the physiological data received at step 908 and baseline user preferences stored in the memory 422. When the food texture classification indicates moderate, firm, or very firm texture, the target chewing cadence may be determined without the decrease applied for soft foods. Firm and very firm foods may naturally slow eating pace due to the chewing effort required, and the target chewing cadence for these foods may be set at or above a baseline target chewing cadence. The target chewing cadence determined at step 914 may be further determined based on the food properties, including the food texture classification, even when the texture does not indicate soft food. From step 914, the method 900 may proceed to step 918.

At step 916, the controller 418 may generate a control signal based on the decreased target chewing cadence determined at step 912 and may transmit the control signal to the gastric vibrational therapy device 416. The control signal generated at step 916 may specify operational parameters for the gastric vibrational therapy device 416 including vibration frequency, vibration amplitude, duty cycle, and stimulation duration. The controller 418 may calculate a deviation between the actual chewing cadence received at step 906 and the decreased target chewing cadence determined at step 912. When the calculated deviation indicates that the chewing cadence exceeds the decreased target chewing cadence, the control signal may specify increased vibration amplitude to provide enhanced mechanical stimulation that compensates for rapid eating of soft foods. The controller 418 may transmit the control signal to the gastric vibrational therapy device 416 via the communication module 426 using a wireless communication protocol such as Bluetooth. The gastric vibrational therapy device 416 may receive the control signal through the GVT communication module 510 and the vibration actuator 502 may deliver non-invasive mechanical stimulation to a gastric region of the user based on the specified operational parameters.

Further, the controller 418 may calculate a deviation of 8 chews per minute between the actual chewing cadence of 38 chews per minute and the decreased target chewing cadence of 30 chews per minute, representing a 27% positive deviation. The controller 418 may generate a control signal specifying an increase in vibration amplitude from 0.5 g-force to 0.7 g-force to compensate for the user eating faster than the target pace for soft foods.

At step 918, the controller 418 may generate a control signal based on the target chewing cadence determined at step 914 and may transmit the control signal to the gastric vibrational therapy device 416. The control signal generated at step 918 may specify operational parameters for the gastric vibrational therapy device 416 based on the standard target chewing cadence determined for non-soft foods. The controller 418 may calculate a deviation between the actual chewing cadence received at step 906 and the target chewing cadence determined at step 914. The controller 418 may transmit the control signal to the gastric vibrational therapy device 416 via the communication module 426 using a wireless communication protocol. The gastric vibrational therapy device 416 may receive the control signal through the GVT communication module 510 and the vibration actuator 502 may deliver non-invasive mechanical stimulation to a gastric region of the user based on the specified operational parameters.

The method 900 may return to step 902 following step 916 or step 918 to continue the continuous monitoring cycle, with the image capture device 402 acquiring additional food images and the chewing detection sensor 406 generating subsequent chewing data for analysis by the controller 418. The method 900 may provide an adaptive approach to gastric satiety therapy by adjusting the target chewing cadence based on food texture classification, with soft foods triggering a decreased target chewing cadence to enhance satiety signaling during consumption of foods that naturally require less chewing effort.

In the context of a non-transitory computer-readable medium storing instructions, the instructions may further cause the processor to receive image data from an image capture device as performed at step 902 and determine food properties from the image data as performed at step 904. The target chewing cadence may be further determined based on the food properties, including the food texture classification determined from the image data. The food properties may comprise a food texture classification that categorizes foods into texture categories based on chewing requirements. The instructions may further cause the processor to decrease the target chewing cadence when the food texture classification indicates a soft food texture as performed at step 912. The decrease of the target chewing cadence for soft food textures may compensate for reduced mechanical satiety stimulation by encouraging slower eating that provides additional time for satiety signals to develop through hormonal and gastric distension pathways.

The adaptive gastric satiety therapy system provides multi-modal integration advantages by combining data from chewing detection sensors, continuous glucose monitors, heart rate variability monitors, sleep tracking devices, activity trackers, and image capture devices. This integration enables more complete representation of factors influencing metabolic responses and eating behavior, achieving prediction accuracy exceeding single-modality approaches and identifying relationships between input factors and metabolic outcomes not apparent from isolated analysis.

The system discovers synergistic interaction effects between input factors through machine learning analysis of historical meal data. Discovered interactions include sleep deprivation combined with elevated stress, high carbohydrate content with certain menstrual cycle phases, social dining context with alcohol consumption, and meal timing with glycemic index. The system applies multiplicative adjustment factors for these interactions, providing more accurate risk assessment when multiple risk factors are present simultaneously.

The system provides individual phenotype recognition through feature importance analysis, classifying users into categories including sleep-sensitive, stress-reactive, cycle-driven, and balanced phenotypes. This classification informs therapeutic protocol personalization, with users receiving increased intervention for factors to which they exhibit elevated sensitivity, improving prediction accuracy and therapeutic effectiveness compared to population-based algorithms.

The system provides non-compliance-dependent efficacy wherein the gastric vibrational therapy device delivers therapeutic benefit through mechanical stimulation independent of user behavioral compliance. When users eat rapidly despite guidance, the device continues activating gastric mechanoreceptors, increasing vagal signaling, and promoting satiety hormone secretion. Compensatory protocols increase intervention intensity when non-compliance is detected, distinguishing this system from passive feedback systems relying entirely on behavioral compliance.

The system provides temporal sophistication through multi-phase intervention including pre-meal priming to pre-activate vagal signaling pathways, intra-meal modulation with real-time parameter adjustment based on detected eating behavior, and post-meal compensation providing sustained satiety support during the postprandial period. This multi-phase approach provides more complete therapeutic coverage compared to single-phase intervention systems.

In one embodiment, the gastric vibrational therapy system may be implemented as a multi-position gastric vibrational therapy system. The multi-position gastric vibrational therapy system may comprise a first gastric vibrational therapy device configured to be positioned over a cardio region of a stomach of a user and a second gastric vibrational therapy device configured to be positioned over a pylorus region of the stomach of the user. The cardio region is located at the upper portion of the stomach near the gastroesophageal junction, while the pylorus region is located at the lower portion of the stomach near the pyloric sphincter. Vagal afferent nerve fibers are distributed throughout both the cardio region and the pylorus region, and stimulation of nerve fibers at both locations may provide enhanced satiety signaling compared to stimulation at a single location.

The controller may be configured to independently control a first operating frequency of the first gastric vibrational therapy device and a second operating frequency of the second gastric vibrational therapy device. Independent control of the operating frequencies enables the system to compensate for differential tissue attenuation between the cardio region and the pylorus region. Tissue attenuation refers to the reduction in mechanical vibration amplitude as the vibration travels through subcutaneous tissue from the skin surface to the underlying gastric wall. The degree of tissue attenuation depends on the thickness and composition of the subcutaneous tissue at each location.

The controller may be configured to deliver a target stimulation frequency in the range of 60-100 Hz to nerve fibers at each region. The target stimulation frequency represents the frequency of mechanical vibration that reaches the vagal afferent nerve fibers in the gastric wall after transmission through the subcutaneous tissue. Research has established that mechanical stimulation in the 60-100 Hz range is effective for activating gastric mechanoreceptors and vagal afferent fibers involved in satiety signaling.

The first operating frequency and the second operating frequency may each be in the range of 80-250 Hz. The operating frequency refers to the frequency at which the gastric vibrational therapy device generates mechanical vibration at the skin surface. The operating frequency is higher than the target stimulation frequency to compensate for frequency-dependent attenuation that occurs as the mechanical vibration travels through subcutaneous tissue. Higher frequencies experience greater attenuation than lower frequencies when traveling through soft tissue, so the operating frequency must be set higher than the desired target stimulation frequency to ensure adequate stimulation reaches the nerve fibers.

The multi-position gastric vibrational therapy system may further comprise a tissue composition sensor configured to measure subcutaneous tissue thickness at the cardio region and at the pylorus region. The tissue composition sensor may comprise an ultrasound transducer, a bioimpedance sensor, or a near-infrared spectroscopy sensor configured to measure tissue thickness and composition. The tissue composition sensor may be integrated into the gastric vibrational therapy devices or may be a separate handheld device used during initial system setup and calibration.

The controller may be configured to receive tissue composition data from the tissue composition sensor indicating subcutaneous tissue thickness at the cardio region and the pylorus region. The controller may calculate a first attenuation factor for the cardio region based on the measured subcutaneous tissue thickness at the cardio region. Subcutaneous tissue thickness inversely correlates with mechanical vibration transmission efficiency, meaning that greater tissue thickness results in greater attenuation of the mechanical vibration. The controller may calculate a second attenuation factor for the pylorus region based on the measured subcutaneous tissue thickness at the pylorus region.

The controller may determine the first operating frequency based on the first attenuation factor to compensate for tissue attenuation and deliver the target stimulation frequency to the cardio region. The controller may determine the second operating frequency based on the second attenuation factor to compensate for tissue attenuation and deliver the target stimulation frequency to the pylorus region. By independently adjusting the operating frequencies based on the measured tissue composition at each location, the system can ensure that the target stimulation frequency is delivered to nerve fibers at both the cardio region and the pylorus region despite differences in subcutaneous tissue thickness between the two locations.

The multi-position gastric vibrational therapy system may further comprise a chewing detection sensor configured to monitor eating behavior of the user and generate chewing data indicative of a chewing cadence. The controller may be configured to modulate the first operating frequency and the second operating frequency based on a deviation between the chewing cadence and a target chewing cadence. When the chewing cadence exceeds the target chewing cadence, the controller may increase the operating frequencies to provide enhanced gastric stimulation that compensates for rapid eating behavior.

The multi-position gastric vibrational therapy system may further comprise a physiological monitoring device configured to acquire physiological data from the user including at least one of glucose levels or heart rate variability metrics. The controller may be configured to determine the target stimulation frequency based on the physiological data. When glucose levels are elevated or heart rate variability metrics indicate sympathetic dominance, the controller may increase the target stimulation frequency to provide enhanced therapeutic intervention.

The system provides circadian adjustments by modifying therapeutic protocols based on meal timing relative to circadian rhythms, with meals during periods of reduced glucose tolerance receiving enhanced therapeutic support. The system accounts for individual circadian phase based on sleep-wake patterns, enabling appropriate adjustments for users with non-standard schedules.

The system provides schedule disruption handling by detecting deviations from established eating patterns and applying enhanced therapeutic intervention to compensate for metabolic effects associated with irregular eating patterns during travel, holidays, or work schedule changes.

The system provides day-of-week pattern learning by identifying systematic variations in eating behavior and metabolic responses across different days, applying enhanced therapeutic intervention on days identified as elevated risk based on learned patterns for proactive intervention.

The adaptive gastric satiety therapy system may be applied across multiple use cases addressing different populations and therapeutic objectives for weight management, metabolic control, and appetite regulation. The system may be applied for weight management in individuals with obesity (BMI of 30 or greater). The mechanical stimulation delivered by the gastric vibrational therapy device may activate vagal afferent pathways that signal fullness, while chewing cadence guidance encourages eating behaviors associated with enhanced satiety hormone secretion. The multi-modal integration enables identification of individual factors contributing to overconsumption and provides personalized intervention addressing those specific factors.

The system may be applied for metabolic control in individuals with type 2 diabetes. The system may reduce postprandial glucose excursions through promotion of slower eating pace, distributing carbohydrate absorption over longer time periods. Integration with the continuous glucose monitor enables real-time detection of glucose dynamics and adjustment of therapeutic intervention based on observed glucose response through glucose-responsive protocol adjustments.

The system may be applied for metabolic control in individuals with prediabetes. The system may reduce postprandial glucose excursions and promote eating behaviors associated with improved insulin sensitivity, helping prevent or delay progression from prediabetes to type 2 diabetes through early intervention.

The system may be applied for appetite regulation in individuals with emotional eating behaviors. Integration with stress detection through the heart rate variability monitor and galvanic skin response sensors enables identification of eating during elevated stress states. The system provides enhanced therapeutic support during stress-related eating episodes to promote satiety signaling and reduce overconsumption.

The system may be applied for individuals with a history of obesity who have achieved weight loss and seek to maintain the reduced weight. The machine learning engine may identify patterns associated with weight regain risk, such as changes in eating pace, meal timing, or physiological indicators, and provide enhanced therapeutic support when weight regain risk indicators are detected.

The system may be applied for individuals who have undergone bariatric surgery and seek additional support for weight management. The mechanical stimulation may activate vagal afferent pathways that complement anatomical changes produced by surgery, while chewing cadence guidance encourages eating behaviors appropriate for altered gastrointestinal anatomy, such as slower eating pace and smaller bite sizes.

The system may integrate with healthcare provider oversight for monitoring therapy compliance and adjusting protocols. Remote monitoring functions enable healthcare providers to access data regarding therapy compliance, eating behavior patterns, physiological responses, and therapeutic outcomes through a healthcare provider portal. Healthcare providers may review and adjust therapeutic protocols including target chewing cadence ranges, gastric vibrational therapy intensity parameters, and therapy duration based on clinical assessment.

The healthcare provider oversight enables identification of patients who may benefit from additional clinical intervention, such as patients exhibiting persistent non-compliance, patients not achieving expected improvements, or patients exhibiting concerning patterns in physiological data. Data transmitted between the system and healthcare provider portals may be encrypted and access restricted to authorized healthcare providers in accordance with applicable privacy regulations.

In various embodiments, the software components described herein are implemented on one or more general-purpose computers, specialized medical devices, cloud-based servers, mobile computing devices, or distributed computing systems. The methods and systems disclosed are embodied in computer-readable instructions stored on non-transitory computer-readable media, including but not limited to solid-state memory, magnetic storage, optical storage, or combinations thereof. The machine learning algorithms and artificial intelligence models described herein are executed on processors including central processing units (CPUs), graphics processing units (GPUs), application-specific integrated circuits (ASICs), field-programmable gate arrays (FPGAs), or neural processing units configured for inference operations.

In some aspects, the satiety enhancement system is configured to comply with applicable regulatory requirements for medical devices, including data privacy and security standards for protected health information. The system implements encryption protocols for data transmission between the gastric vibrational therapy device, user devices, and servers. In some cases, the system incorporates authentication mechanisms to verify the identity of users and healthcare personnel accessing physiological data and therapy protocols.

The physiological data processing and chewing detection methods described herein are performed locally on the wearable satiety enhancement system, on the electronic device, on a remote server, or through a combination of local and remote processing. In some embodiments, the distribution of processing tasks is dynamically adjusted based on available computational resources, network connectivity, power constraints, or latency requirements for time-sensitive therapeutic interventions.

The machine learning models described herein, including those for glucose prediction and eating behavior analysis, are trained using supervised learning, unsupervised learning, reinforcement learning, or combinations thereof. In some aspects, the models are pre-trained on population-level physiological data and subsequently fine-tuned using individual user data to establish personalized baselines and thresholds. The training data includes labeled examples of normal eating patterns and various metabolic conditions to enable classification of detected deviations.

In some cases, the system is configured to operate in accordance with interoperability standards for health information exchange, enabling integration with electronic health record systems, clinical decision support tools, and healthcare provider communication platforms. The therapy protocol transmission mechanisms support multiple communication protocols and are configured to route notifications based on the availability and preferences of designated healthcare personnel.

Reference throughout this specification to "an example" means that a particular feature, structure, or characteristic described in connection with the example is included in at least one embodiment of the present invention. Thus, appearances of the phrase "in an example" in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

Those having skill in the art will appreciate that many changes may be made to the details of the above-described embodiments without departing from the underlying principles of the invention. The scope of the present invention should, therefore, be determined only by the following claims.

What is claimed is:

1. A satiety enhancement system for adaptive control of gastric satiety therapy, the satiety enhancement system comprising:
a gastric vibrational therapy device configured to deliver non-invasive mechanical stimulation to a gastric region of a user;
a chewing detection sensor configured to monitor eating behavior of the user and generate chewing data indicative of a chewing cadence;
a physiological monitoring device configured to acquire physiological data from the user; and
a controller in communication with the gastric vibrational therapy device, the chewing detection sensor, and the physiological monitoring device, the controller configured to:
receive the chewing data from the chewing detection sensor;
receive the physiological data from the physiological monitoring device;
determine a target chewing cadence based on the physiological data;
calculate a deviation between the chewing cadence and the target chewing cadence; and
modulate operational parameters of the gastric vibrational therapy device based on the calculated deviation.

2. The satiety enhancement system of claim 1, wherein the chewing detection sensor comprises at least one of an acoustic sensor, an inertial measurement unit, an electromyographic sensor, or a strain sensor.

3. The satiety enhancement system of claim 2, wherein the acoustic sensor is configured to detect chewing sounds and the controller is configured to identify chewing events from the detected chewing sounds using signal processing algorithms.

4. The satiety enhancement system of claim 1, wherein the physiological monitoring device comprises a continuous glucose monitor configured to measure glucose levels of the user and a heart rate variability monitor configured to measure heart rate variability metrics indicative of autonomic nervous system state.

5. The satiety enhancement system of claim 4, wherein the controller is further configured to calculate a glucose-based adjustment factor based on the glucose levels and apply the glucose-based adjustment factor to the operational parameters of the gastric vibrational therapy device.

6. The satiety enhancement system of claim 1, wherein the operational parameters of the gastric vibrational therapy device comprise at least one of vibration frequency, vibration amplitude, duty cycle, or stimulation duration, and wherein the controller is configured to increase the vibration amplitude when the deviation indicates the chewing cadence exceeds the target chewing cadence.

7. The satiety enhancement system of claim 1, further comprising an image capture device configured to acquire images of food, and an image analysis module configured to determine food properties from the acquired images, wherein the food properties comprise at least one of food texture classification, estimated caloric content, macronutrient composition, or glycemic index, and wherein the controller is further configured to determine the target chewing cadence based on the food properties.

8. The satiety enhancement system of claim 1, wherein the controller is further configured to receive behavioral data including at least one of meal timing patterns, eating duration, compliance history with target chewing cadence, or social context indicators, and wherein the target chewing cadence is further determined based on the behavioral data.

9. The satiety enhancement system of claim 1, wherein the physiological monitoring device further comprises at least one of a skin temperature sensor configured to measure skin temperature indicative of autonomic nervous system state, an abdominal distention sensor configured to measure changes in abdominal circumference indicative of gastric filling, or a gut peptide sensor configured to measure levels of at least one of glucagon-like peptide-1 (GLP-1), peptide YY (PYY), cholecystokinin (CCK), ghrelin, or leptin.

10. The satiety enhancement system of claim 1, further comprising a safety manager configured to detect vehicle operation and automatically suspend artificial intelligence (AI)-controlled operation of the gastric vibrational therapy device during detected vehicle operation.

11. The satiety enhancement system of claim 1, further comprising:
an electronic device in communication with the controller, the electronic device configured to generate a control signal to modulate the operational parameters of the gastric vibrational therapy device based on the calculated deviation; and
a server in communication with the electronic device, the server configured to:
store historical records including meal events and physiological state data;
execute machine learning models using the historical records to generate predictions; and
transmit personalized therapy protocols to the controller for implementation by the satiety enhancement system.

12. A method for adaptive control of gastric satiety therapy, the method comprising the steps of:
delivering, by a gastric vibrational therapy device, non-invasive mechanical stimulation to a gastric region of a user;
monitoring, by a chewing detection sensor, eating behavior of the user to generate chewing data indicative of a chewing cadence;
acquiring, by a physiological monitoring device, physiological data from the user;
receiving, by a controller in communication with the gastric vibrational therapy device, the chewing detection sensor, and the physiological monitoring device, the chewing data from the chewing detection sensor;
receiving, by the controller, the physiological data from the physiological monitoring device;
determining, by the controller, a target chewing cadence based on the physiological data;
calculating, by the controller, a deviation between the chewing cadence and the target chewing cadence; and
modulating, by the controller, operational parameters of the gastric vibrational therapy device based on the calculated deviation.

13. The method of claim 12, wherein monitoring eating behavior comprises detecting chewing sounds using an acoustic sensor and identifying chewing events from the detected chewing sounds using signal processing algorithms.

14. The method of claim 12, wherein acquiring physiological data comprises measuring glucose levels of the user using a continuous glucose monitor, and wherein the method further comprises calculating a glucose-based adjustment factor based on the glucose levels and applying the glucose-based adjustment factor to the operational parameters of the gastric vibrational therapy device.

15. The method of claim 12, further comprising detecting an anticipatory glucose decline pattern from the physiological data, and preemptively activating the gastric vibrational therapy device to initiate pre-meal priming before the user begins eating.

16. The method of claim 12, further comprising:
acquiring image data of food to be consumed by the user;
determining from the image data that the food has a soft food texture associated with reduced mechanical satiety stimulation compared to a firm food texture;
in response to determining the soft food texture, decreasing the target chewing cadence below a baseline chewing cadence to compensate for the reduced mechanical satiety stimulation by extending eating duration to allow time for non-mechanical satiety signals to develop; and
providing eating pace guidance based on the decreased target chewing cadence.

17. A multi-position gastric vibrational therapy system comprising:
a first gastric vibrational therapy device configured to be positioned over a cardio region of a stomach of a user;
a second gastric vibrational therapy device configured to be positioned over a pylorus region of the stomach of the user; and
a controller in communication with the first gastric vibrational therapy device and the second gastric vibrational therapy device, the controller configured to independently control a first operating frequency of the first gastric vibrational therapy device and a second operating frequency of the second gastric vibrational therapy device based on differential tissue attenuation between the cardio region and the pylorus region to deliver a target stimulation frequency in the range of 60-100 Hz to nerve fibers at each region, wherein the first operating frequency and the second operating frequency are each in the range of 80-250 Hz.

18. The multi-position gastric vibrational therapy system of claim 17, further comprising:
a tissue composition sensor configured to measure subcutaneous tissue thickness at the cardio region and at the pylorus region;
wherein the controller is further configured to:
receive tissue composition data from the tissue composition sensor indicating subcutaneous tissue thickness at the cardio region and the pylorus region;
calculate a first attenuation factor for the cardio region based on the measured subcutaneous tissue thickness at the cardio region;
calculate a second attenuation factor for the pylorus region based on the measured subcutaneous tissue thickness at the pylorus region;
determine the first operating frequency based on the first attenuation factor to compensate for tissue attenuation and deliver the target stimulation frequency to the cardio region; and
determine the second operating frequency based on the second attenuation factor to compensate for tissue attenuation and deliver the target stimulation frequency to the pylorus region.

19. The multi-position gastric vibrational therapy system of claim 17, further comprising:
a chewing detection sensor configured to monitor eating behavior of the user and generate chewing data indicative of a chewing cadence; and
wherein the controller is further configured to modulate the first operating frequency and the second operating frequency based on a deviation between the chewing cadence and a target chewing cadence.

20. The multi-position gastric vibrational therapy system of claim 17, further comprising:
a physiological monitoring device configured to acquire physiological data from the user including at least one of glucose levels or heart rate variability metrics;
wherein the controller is further configured to determine the target stimulation frequency based on the physiological data.
